(19) United States
(12) Patent Application Publication
(10) Pub. No.: US 2024/0000161 A1
(43) Pub. Date: Jan. 4, 2024

(54) CESSATION COMPANION DIAGNOSTICS ANALYZER METHOD AND DEVICES

(71) Applicant: CUTTING EDGE EHEALTH SOLUTIONS, LLC, Northridge, CA (US)

(72) Inventors: Arshad H Khan, St. James (BB); Edmond DeFrank, Northridge, CA (US); Nicolas DeFrank, Northridge, CA (US); Antonio DeFrank, Northridge, CA (US)

(21) Appl. No.: 17/857,097

(22) Filed: Jul. 4, 2022

(51) Int. Cl. A24F 47/00; A61B 5/00; G16H 40/63

(52) U.S. Cl. CPC A24F 47/00; A61B 5/6803; A61B 5/002; A61B 5/4806; G16H 40/63

(57) ABSTRACT

The embodiments disclose a cessation mobile app including a user self-assessment module coupled to the cessation mobile app configured to collect user data, a vital signs module wirelessly coupled to the cessation mobile app configured to monitor and collect vital signs data from a user, a cessation companion diagnostic module coupled to the cessation mobile app configured to collect and monitor cessation intervention products data, and an analyzer module coupled to the cessation mobile app configured to analyze the user data, vital signs data, and cessation intervention products data to provide customized suggestions to the user on the cessation intervention products best suited for the user.

### BACKGROUND

The cessation of nicotine tobacco smoking products is difficult since nicotine is an addictive chemical. Users of nicotine tobacco smoking products also face a habitual use pattern that is difficult to change. Social behavior, stress relief, and other conditions add to the cessation planning and ability to quit the nicotine tobacco smoking products use and habits.

### BRIEF DESCRIPTION OF THE DRAWINGS

FIG. 1 shows a block diagram of an overview flow chart of a cessation mobile app connection of one embodiment.

FIG. 2 shows a block diagram of an overview flow chart of a cessation mobile app of one embodiment.

FIG. 3 shows for illustrative purposes only an example of smoke detection sensor of one embodiment.

FIG. 4 shows for illustrative purposes only an example of cessation mobile app modules of one embodiment.

FIG. 5 shows for illustrative purposes only an example of support group help auto-text message of one embodiment.

FIG. 6 shows for illustrative purposes only an example of nicotine tobacco cessation questionnaire module of one embodiment.

FIG. 7 shows for illustrative purposes only an example of nicotine tobacco cessation self-assessment module of one embodiment.

FIG. 8A shows a block diagram of an overview flow chart of user smart phone sensor modules of one embodiment.

FIG. 8B shows a block diagram of an overview flow chart of nicotine tobacco cessation vital signs module of one embodiment.

FIG. 9 shows a block diagram of an overview flow chart of nicotine tobacco cessation artificial intelligence module of one embodiment.

FIG. 10 shows for illustrative purposes only an example of a cessation digital wearable of one embodiment.

FIG. 11 shows a block diagram of an overview flow chart of a cessation questionnaire module based on vital signs of one embodiment.

FIG. 12 shows a block diagram of an overview of smoking health risks of one embodiment.

FIG. 13 shows a block diagram of an overview of smoking disrupts circadian rhythms of one embodiment.

FIG. 14 shows a block diagram of an overview of smoking cessation circadian rhythms temporary conditions and health benefits of one embodiment.

FIG. 15 shows a block diagram of an overview of smoking cessation circadian rhythms benefits of one embodiment.

FIG. 16 shows a block diagram of an overview of smoking cessation circadian rhythms monitoring of one embodiment.

FIG. 17 shows a block diagram of an overview of smoking cessation circadian rhythms re-entrainment of one embodiment.

FIG. 18 shows a block diagram of an overview of cessation of nicotine tobacco smoking habits of one embodiment.

FIG. 19 shows a block diagram of an overview of cessation intervention companion diagnostic analysis of one embodiment.

FIG. 20 shows a block diagram of an overview of prescribed medications delivery systems of one embodiment.

FIG. 21 shows a block diagram of an overview of types of nicotine replacement therapy of one embodiment.

FIG. 22 shows a block diagram of an overview of possible cessation tools and medications side effects of one embodiment.

FIG. 23 shows a block diagram of an overview of gathering user data of one embodiment.

FIG. 24 shows a block diagram of an overview of virtual reality games to quit smoking of one embodiment.

### DETAILED DESCRIPTION OF THE INVENTION

In the following description, reference is made to the accompanying drawings, which form a part hereof, and which is shown by way of illustration a specific example in which the invention may be practiced. It is to be understood that other embodiments may be utilized, and structural changes may be made without departing from the scope of the present invention.

### General Overview

It should be noted that the descriptions that follow, for example, in terms of a cessation mobile app method and devices is described for illustrative purposes and the underlying system can apply to any number and multiple types of plant materials. In one embodiment of the present invention, the cessation mobile app method and devices can be configured using cigarettes. The cessation mobile app method and devices can be configured to operate on multiple mobile device operating systems and can be configured to include installation on multiple mobile digital devices using the present invention. The terms “smoker”, “ex-smoker” and “user” are used herein interchangeably without any change in meaning.

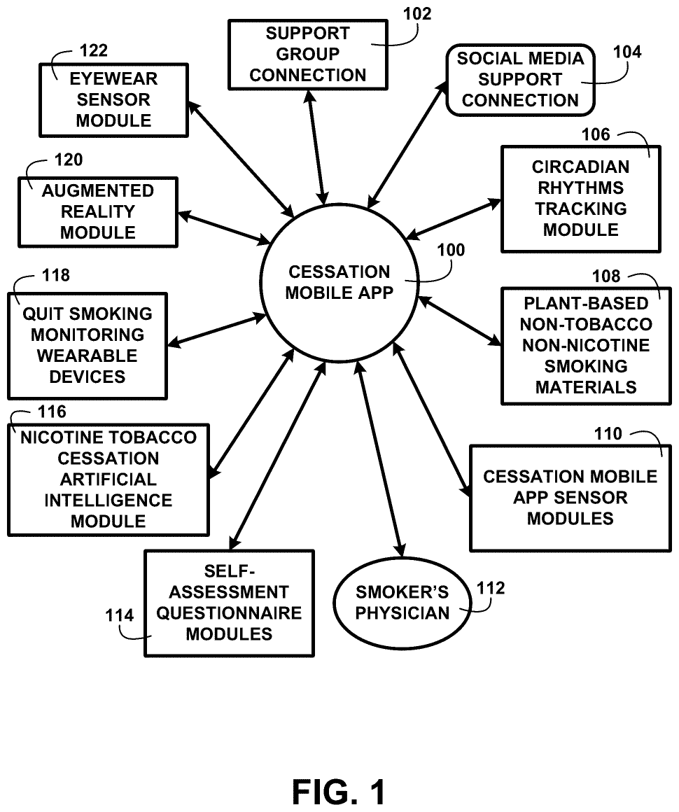

FIG. 1 shows a block diagram of an overview flow chart of cessation mobile app connections of one embodiment. FIG. 1 shows in one embodiment, for example a cessation mobile app 100 connections are used to form a holistic approach to quitting smoking. The cessation mobile app 100 provides a smoker direct access through a support group connection 102 and a social media support connection 104 to assist the smoker's efforts in quitting tobacco and nicotine smoking.

The nicotine addiction is in part difficult to quit due to circadian rhythms adapting to the smoker's habits and environmental cues. In one embodiment, for example, smoking after eating, driving to work, and circadian clock adherence to the smoker's smoking routine. Circadian rhythms tracking module 106 coupled to the cessation mobile app 100 provides the cessation mobile app 100 with data on when those routine cues are in action and alerting the smoker to change the routine environment to break the timing and conditions.

The cessation mobile app 100 provides availability information for smokers to find the plant-based non-tobacco non-nicotine smoking materials 108 that provide assistance by first eliminating the harsh chemicals in tobacco with the nicotine fueling the addiction. The plant-based non-tobacco non-nicotine smoking materials 108 further provide an alternative that replaces the physical and emotional dependence of the very act of lighting up a cigarette. This lessens the initial difficulties in quitting to the drug withdrawal and safely substitutes an alternative for the physical and emotional habitual routine.

Cessation mobile app sensor modules 110 provide sensor detection data and visual confirmation of smoke around the smoker and sounds data during sleep to provide progress in the cessation efforts. The cessation mobile app 100 provides analysis of all sensor data transmitted to the cessation mobile app 100 to provide progress or the lack thereof in the cessation efforts.

A smoker's physician 112 can be authorized by the smoker to receive the sensor data from the cessation mobile app 100 to check progress as it relates to existing smoker health conditions and preventative treatments and procedures. The data also provides the smoker with the necessary data to see their progress in reducing health risks, improved sleep, controlling eating rather than the desire for nicotine controlling their appetite, gaining energy and activity, and realizing a sense of accomplishment.

The smoker supplies their own assessment of progress using the self-assessment questionnaire modules 114 administered by the cessation mobile app 100. The responses to the questionnaires inquiries is gathered textually and verbally including live streaming of the smoker during the inquiry. A nicotine tobacco cessation artificial intelligence module 116 coupled to the cessation mobile app 100 analyzes the smoker's responses and demeanor to compare to the sensor data and truthfulness of the smoker based on visual clues and voice inflection.

Sensors are embedded into quit smoking monitoring wearable devices 118 to detect and measure vital signs directly connected to the circadian rhythms. The quit smoking monitoring wearable devices 118 are wirelessly coupled to the cessation mobile app 100 to transmit the detected and measured data. The data collected can assist the smoker in making changes to prior habitual routines. The changes are helpful to gain improved sleep cycles for restful slumber. The data also assists the smoker in altering the circadian rhythms to adjust more quickly to nicotine withdrawal and progress on the cessation path. An augmented reality module 120 in cessation mobile app 100 provides data in recognizing the smoking habit conditions surrounding the smoker and alerting the smoker to stop and make a shift in the activity to avoid smoking.

An eyewear sensor module 122 provides data on the smoker's sleep activities and transmits the data to the cessation mobile app 100. In one embodiment, for example the smoker is demonstrating rapid eye movement (REM).

### Cessation Mobile App:

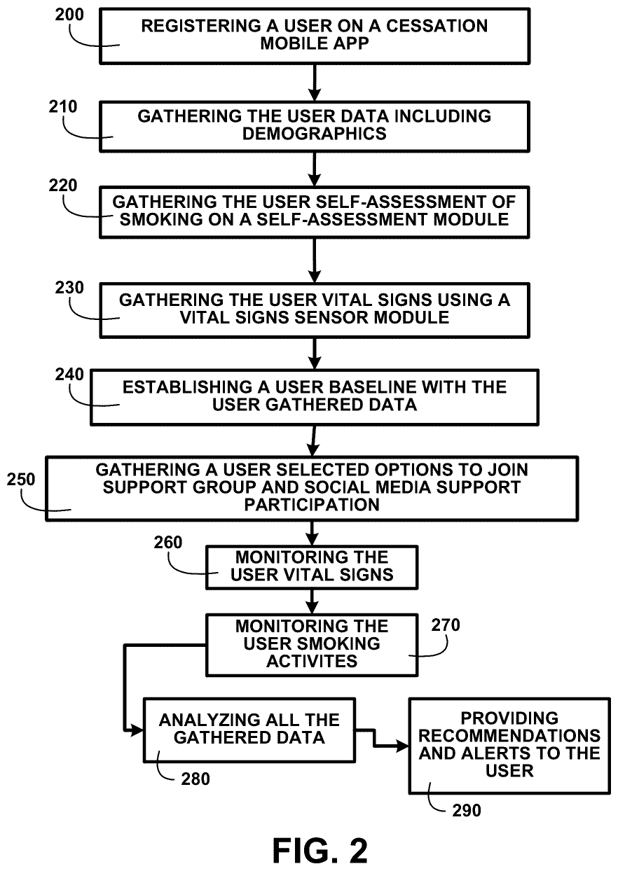

FIG. 2 shows a block diagram of an overview flow chart of a cessation mobile app of one embodiment. FIG. 2 shows registering a user on a cessation mobile app 200. Gathering the user's data including demographics 210. Gathering the user's self-assessment of smoking on a self-assessment module 220. Gathering the user's vital signs using a vital signs sensor module 230. Establishing a user's baseline with the user gathered data 240. Gathering a user's selected option of joining a support group and social media support participation 250. Monitoring the user's vital signs 260. Monitoring the user's smoking activities 270. Analyzing all the gathered data 280. Providing recommendations and alerts to the user 290. Gathering user data includes public data and from social media.

A cessation mobile app 100 of FIG. 1 to assist the smoker in breaking the nicotine tobacco habit is provided to aid a smoker in this instance in the cessation of nicotine and tobacco product use. The cessation mobile app 100 of FIG. 1 includes modules to gather information and data to anticipate when the smoker may run into problems sticking to their cessation efforts.

One such module is a nicotine tobacco cessation questionnaire module. The nicotine tobacco cessation questionnaire module displays questions related to the smoker's habit background. A nicotine tobacco cessation self-assessment module displayed questions related to the current smoker habit conditions that have triggered the use of nicotine tobacco products. The questions displayed can be answered verbally by the smoker. The smoker's verbal responses are transcribed and recorded in the cessation mobile app 100 of FIG. 1 and transmitted to a cessation mobile app 100 of FIG. 1 network for recording on databases.

A nicotine tobacco cessation education module provides information for the smoker on the dangers of nicotine, their effect on their health, the benefits of ceasing nicotine and tobacco use, and suggestions on dealing with nicotine withdrawal and day-to-day changes in activities to reduce stress and other triggers that lead to nicotine and tobacco use. The urge for tobacco is likely to be strongest in the situations where you smoked tobacco most often, such as at parties or bars, or while feeling stressed or sipping coffee. The cessation mobile app 100 of FIG. 1 identifies those trigger situations and suggests a plan to avoid them entirely or get through them without using tobacco. It reminds the smoker not to set themselves up for a smoking relapse. If a smoker usually smoked while talking on the phone, for instance, they can keep a pen and paper nearby to occupy themselves with doodling rather than smoking.

The smoker is encouraged to enlist the support of family, friends, and other smokers wanting to quit nicotine and tobacco use. A nicotine tobacco cessation support group module is helpful in creating a support group and keeping communications with them easier. This includes automatically sending support group members the smoker's progress and rapidly connecting with them when help is asked for by the smoker.

A nicotine tobacco cessation progress analysis module provides the smoker with graphs, charts, and data on how their cessation activities are proceeding. A nicotine tobacco cessation smoke detection sensor module identifies smoke content, specifically nicotine, in smoke in and around the smoker's cell phone. This allows the smoker to be aware if they have regressed and are in the company of others smoking nicotine tobacco products. Nicotine tobacco cessation remote vital signs sensor connectivity module is wirelessly coupled to vital signs wearables the smoker may be wearing. In one embodiment, for example tracking vital signs is data that shows when a smoker's blood pressure and heart rate increase with nicotine tobacco use and even second-hand smoke of others.

A nicotine tobacco cessation artificial intelligence module tracks smoker activities and gathers daily data along with the questionnaire data is processed with machine learning to provide the smoker with current conditions and circumstances that can be detrimental to their cessation goal. The cessation mobile app 100 of FIG. 1 is assisting the smoker in dealing with nicotine withdrawal and suggesting habit changes for not returning to their old smoking products in one embodiment.

### DETAILED DESCRIPTION

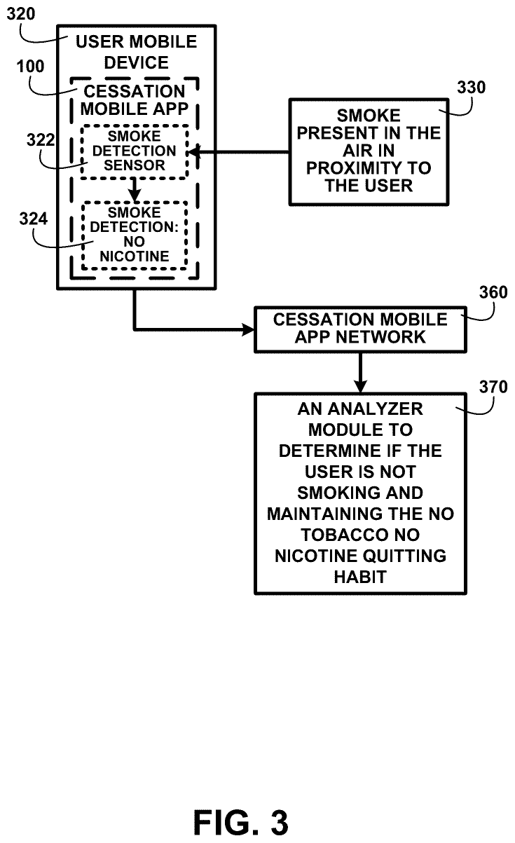

FIG. 3 shows for illustrative purposes only an example of smoke detection sensor of one embodiment. FIG. 3 shows a user mobile device 320 with the cessation mobile app 100. A smoke detector sensor 322 detects smoke present in the air in proximity to the user 330. A smoke detection of no nicotine 324 and the detection data is transmitted to a cessation mobile app network 360. In one embodiment, for example the cessation mobile app network 360 includes an analyzer module to determine if the user is not smoking and maintaining the no tobacco no nicotine quitting habit 370.

In one embodiment, for example gathering photographs and video of the user smoking with a cigarette in the hand and in the mouth and exhaling smoke is for comparative machine learning and analysis. In one embodiment, for example gathering chemical sensor data is used for analysis of other chemicals in tobacco smoke. In one embodiment, for example The smoker's cell phone with the cessation mobile app 100 uses the smoke detection sensor and finds a result of the smoke detection with no nicotine. This means although the smoker is smoking it is not a nicotine tobacco cigarette and may be an alternative cessation intervention plant-based non-tobacco non-nicotine smoking materials 108 of FIG. 1. The cessation mobile app 100 cellular transmissions can be relayed to a support group. In one embodiment, for example an automatic support group smoker nicotine tobacco cessation progress module report to the members of the smoker's support group shares the smoker is maintaining the no tobacco no nicotine cessation effort.

The cessation mobile app 100 cellular transmissions are sent to the network. The cessation mobile app network includes at least one digital server, a plurality of databases, at least one digital processor, cessation mobile app 100 installed on a network computer, and artificial intelligence with machine learning devices for analyzing and machine learning about the smoker's actions related to the cessation of nicotine and tobacco products of one embodiment.

### Cessation Mobile App Modules:

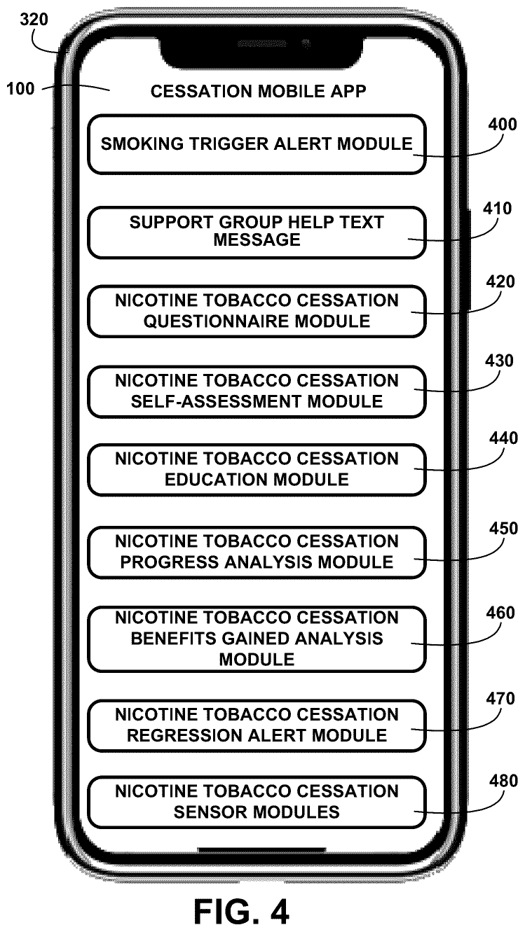

FIG. 4 shows for illustrative purposes only an example of cessation mobile app modules of one embodiment. FIG. 4 shows the user mobile device 320 with the cessation mobile app 100. The cessation mobile app 100 includes a smoking trigger alert module 400, support group help text message 410, nicotine tobacco cessation questionnaire module 420, nicotine tobacco cessation self-assessment module 430, nicotine tobacco cessation education module 440, nicotine tobacco cessation progress analysis module 450, nicotine tobacco cessation benefits gained analysis module 460, nicotine tobacco cessation regression alert module 470, and a nicotine tobacco cessation sensor modules 480.

In one embodiment, for example the smoker has “smoking trigger alerts” displayed on the cessation mobile app 100. The trigger alerts remind the smoker of the times and conditions that have triggered smoking nicotine and tobacco products over time. The trigger alert reminders assist the smoker to maintain their resolve to not regress and continue their cessation of nicotine and tobacco product use. In one embodiment, for example the trigger conditions include 7:15 am to 8:15 am, driving to work, walking upstairs to your office, after lunch, and driving from work. The smoker is also suggested to use a squeeze ball to reduce stress which can trigger nicotine tobacco use. In one embodiment, for example other trigger conditions include after dinner and 9:00 pm to 9:45 pm. The cessation mobile app 100 also displays feature modules the smoker may select to add additional and update information. In one embodiment, for example the cessation mobile app 100 displays tap buttons for support group help auto-text message, nicotine tobacco cessation questionnaire module, nicotine tobacco cessation self-assessment module, nicotine tobacco cessation education modules, nicotine tobacco cessation progress analysis module, nicotine tobacco cessation benefits gained analysis module, nicotine tobacco cessation regression alert module, and nicotine tobacco cessation sensor modules.

### Support Group Help Auto-Text Message:

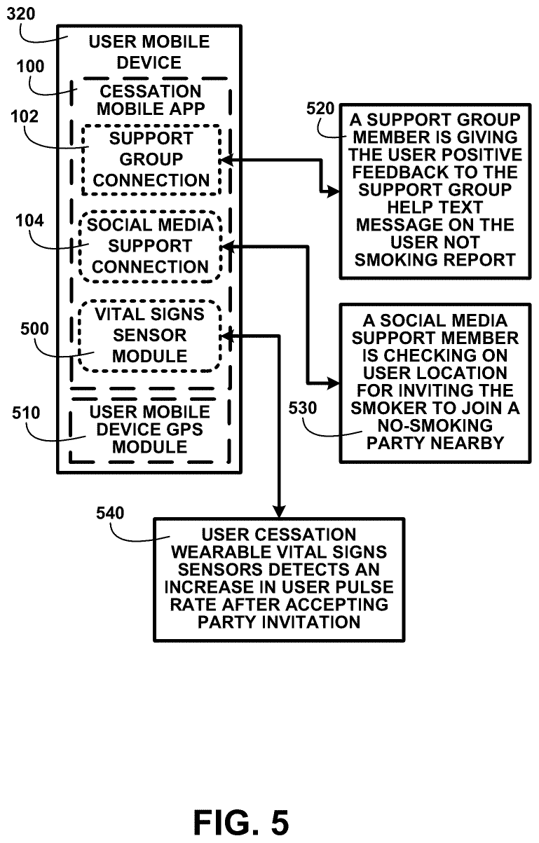

FIG. 5 shows for illustrative purposes only an example of support group help auto-text message of one embodiment. FIG. 5 shows the user mobile device 320 with the cessation mobile app 100. In one embodiment, for example the cessation mobile app 100 includes a support group connection 102, social media support connection 104, vital signs sensor module 500, and user mobile device GPS module 510. In one embodiment, for example a support group member is giving the user positive feedback to the support group help text message on the user not smoking report 520. A social media support member is checking on user location for inviting the smoker to join a no-smoking party nearby 530. A user cessation wearable vital signs sensor detects an increase in user pulse rate after accepting party invitation 540 indicating positive excitement to be joining the party of one embodiment.

### Nicotine Tobacco Cessation Questionnaire Module:

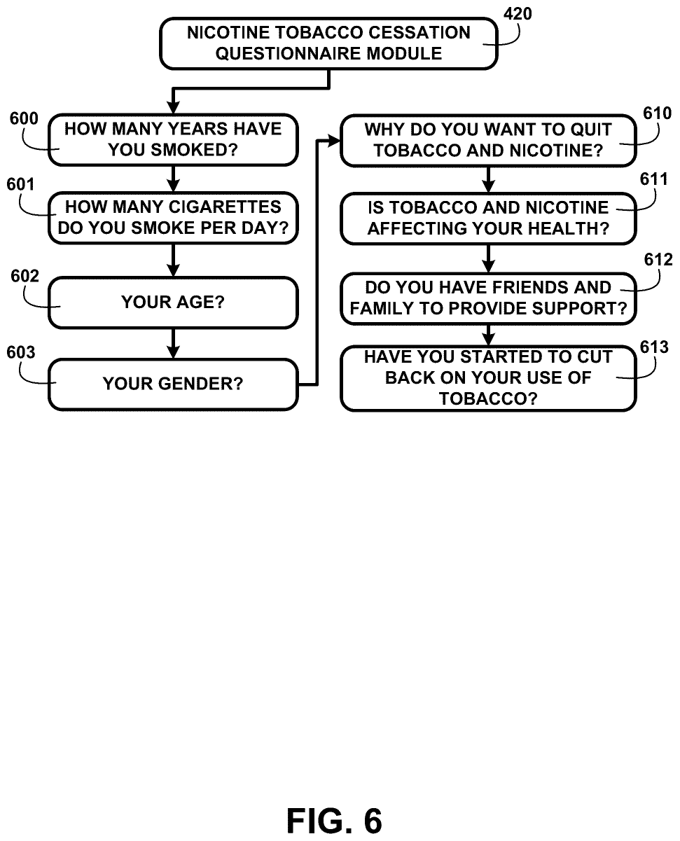

FIG. 6 shows for illustrative purposes only an example of nicotine tobacco cessation questionnaire module of one embodiment. FIG. 6 shows the nicotine tobacco cessation questionnaire module 420. In one embodiment, for example the question includes “How many years have you smoked?” 600. “How many cigarettes do you smoke per day?” 601. “Your age?” 602. “Your gender?” 603. “Why do you want to quit tobacco and nicotine?” 610. “Is tobacco and nicotine affecting your health?” 611. “Do you have friends and family to provide support?” 612. “Have you started to cut back on your use of tobacco?” 613.

The last question shows whether the smoker has a proactive attitude in the cessation of nicotine and tobacco. The cessation mobile app 100 of FIG. 1 displays the nicotine tobacco cessation questionnaire module 420 questions and the smoker may verbally respond to the questions. The verbal responses are transcribed by the cessation mobile app 100 of FIG. 1 and recorded on the smoker's cell phone and transmitted to the network plurality of databases of one embodiment.

### Nicotine Tobacco Cessation Self-Assessment Module:

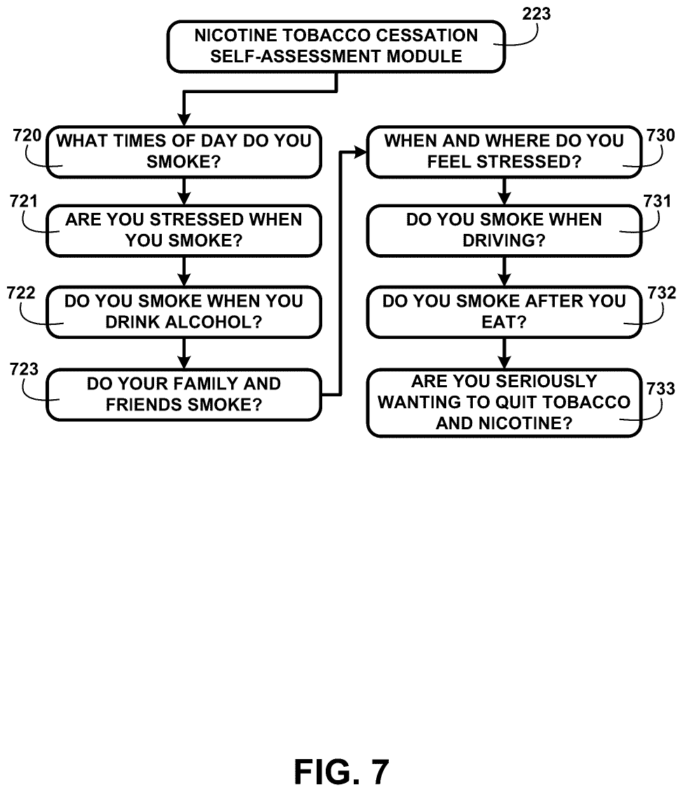

FIG. 7 shows for illustrative purposes only an example of nicotine tobacco cessation self-assessment module of one embodiment. FIG. 7 shows the nicotine tobacco cessation self-assessment module 114. A smoker's self-assessment is useful in identifying the smoker's current habit level and specific conditions in which the smoker's urge to smoke is higher. The nicotine tobacco cessation self-assessment module 114 is accessed by the smoker on the cessation mobile app 100 of FIG. 1.

The cessation mobile app 100 of FIG. 1 displays the nicotine tobacco cessation self-assessment module 114 questions and the smoker verbally responds to the questions. The verbal responses are transcribed by the cessation mobile app 100 of FIG. 1 and recorded on the smoker's cell phone and transmitted to the network plurality of databases.

In one embodiment, for example the questions include “What times of day do you smoke?” 720; “Are you stressed when you smoke?” 721; “Do you smoke when you drink alcohol?” 722; “Do your family and friends smoke?” 723; “When and where do you feel stressed?” 730; “Do you smoke when driving?” 731; and “Do you smoke after you eat?” 732. In one embodiment, for example the last question “Are you seriously wanting to quit tobacco and nicotine?” 733 is also evaluated by a voice inflection module and analyzed to assess the conviction of the smoker in quitting. This is used to increase as needed the encouragement and suggestions to the smoker to strengthen their belief that they can cease nicotine and tobacco use.

### User Smart Phone Sensor Modules:

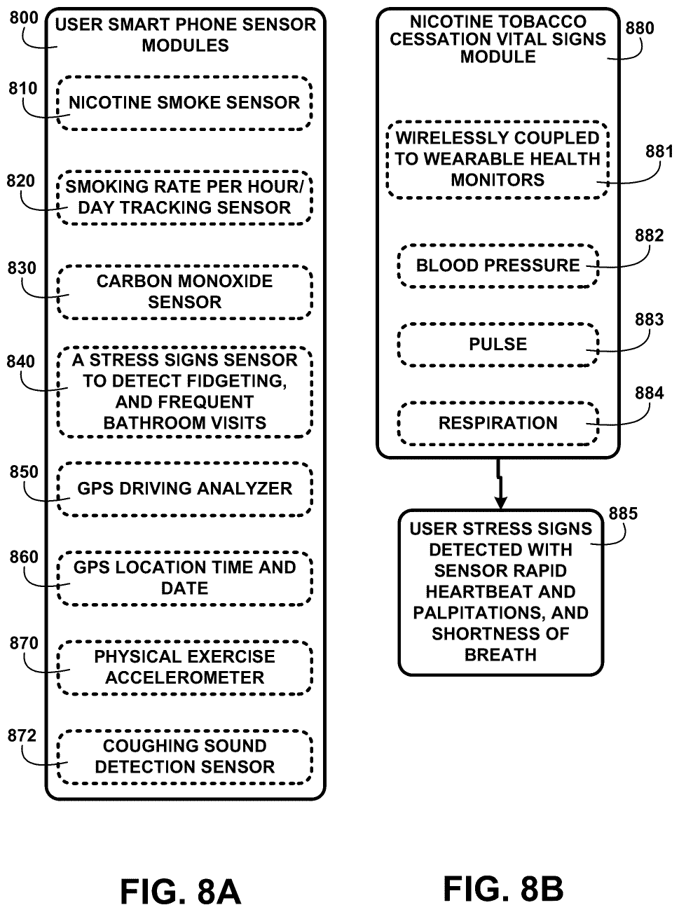

FIG. 8A shows a block diagram of an overview flow chart of user smartphone sensor modules of one embodiment. FIG. 8A shows user smartphone sensor modules 800. In one embodiment, for example the user smartphone sensor modules 800 include monitoring smoking by the smoker, their physical condition before, during, and after smoking, the smoker's location, and time of day, activities, and person in proximity to the smoker. These are conditions and circumstances related to the smoking triggers. In one embodiment, for example the nicotine tobacco cessation sensor modules include a nicotine smoke sensor 810, smoking rate per hour/day tracking sensor 820, carbon monoxide sensor 830, stress signs sensor fidgeting, frequent bathroom visits 840, GPS driving analyzer 850, GPS location time and date 860, physical exercise accelerometer 870, and a coughing sound detection sensor 872 of one embodiment.

The determination of a person in proximity to the smoker provides data for multiple purposes. In one embodiment, for example if a person is smoking in proximity to the smoker, the smoke detector sensor will detect the smoke and activate a camera on the smoker's smartphone to gather confirmation data to determine if it is the user or the person adjacent to the user that is smoking. In another embodiment, the person detected in proximity to the smoker may be someone that previously was determined to be a trigger to cause the user to smoke. In yet another embodiment the person adjacent may be a support member interacting with the smoker to encourage the smoker to maintain the no-smoking status.

In one embodiment, for example the nicotine tobacco cessation sensor modules include a stress signs sensor to detect fidgeting, and frequent bathroom visits 840, when sensing data positively indicates a determination that the smoker may be approaching a trigger to smoke a cigarette. The user's smartphone may text a support member to call the smoker in order to calm down the smoker and encourage them to avoid smoking. The support member may ask the smoker what is the cause of the stress. Sometimes simply talking about a situation can unburden the smoker and have a calming effect. In one embodiment, for example the GPS driving analyzer 850 uses the speed detected by the physical exercise accelerometer 870 to determine if the smoker is driving safely or erratically. When the smoker has stopped a support member may call the smoker to assess the reason for the erratic behavior. The nicotine tobacco cessation sensor modules 880 vital signs monitoring can determine the physical condition of the smoker. The smoker may be feeling anxiety due to a rapid pulse and fast respiration being detected. A support member receiving a text from the user's smartphone may call and check-in with the smoker to determine what is occurring to cause anxiety or other reasons for the high pulse and respiration. The support member provides a backstop for the smoker to let the smoker know they are not going through cessation alone. The support member can remind the smoker that in time the cause may fade as the nicotine is lessened in their body and the benefits being gained far outweigh the temporary discomforts of one embodiment.

### Nicotine Tobacco Cessation Vital Skins Module:

FIG. 8B shows a block diagram of an overview flow chart of nicotine tobacco cessation vital signs module of one embodiment. FIG. 8B shows the nicotine tobacco cessation sensor modules 880 are wirelessly coupled to wearable health monitors 881 of the smoker. In one embodiment, for example the smokers vital signs data gathered include for example blood pressure 882, pulse 883, respiration 884, and user stress signs detected with sensor rapid heartbeat and palpitations, and shortness of breath 885. The wirelessly coupled to wearable health monitors 881 detect and monitor the sensor data on a continuous basis in one embodiment. The wirelessly coupled to wearable health monitors 881 detects and monitors the sensor data on a predetermined time interval set by the user in another embodiment. An on-call operation of the wearable sensor is available in another embodiment wherein the user may push a button to activate the sensors for a predetermined period of time and then shuts them off. The sensor data is transmitted to the cessation mobile app 100 of FIG. 1 where the cessation mobile app 100 of FIG. 1 analyzes the sensor data and determines if the sensor readings show an abnormality or anomaly. In one embodiment, for example the sensor readings analysis also determines if the user's vital signs are within medically acceptable ranges and does not show any signs of stress or other condition that may be potentially dangerous to the user of one embodiment.

### Nicotine Tobacco Cessation Artificial Intelligence Module:

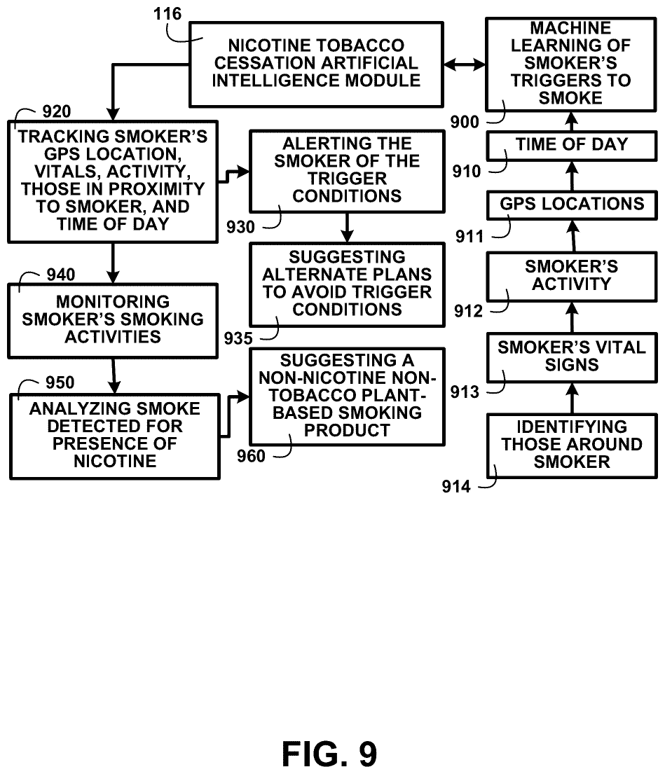

FIG. 9 shows a block diagram of an overview flow chart of nicotine tobacco cessation artificial intelligence module of one embodiment. FIG. 9 shows the nicotine tobacco cessation artificial intelligence module 116. In one embodiment, for example machine learning of smoker's triggers to smoke 900 includes analyzing data gathered, for example, time of day 910, GPS locations 911, smoker's activity 912, smoker's vital signs 913, and identifying those around smoker 914. The learning and analysis include tracking smokers' GPS location, vitals, activity, those in proximity to smokers, and time of day 920. The analyzed data is used for identifying trigger conditions and alerting the smoker of the trigger conditions 930. In one embodiment, for example machine learning includes suggesting alternate plans to avoid trigger conditions 935. Monitoring smoker's smoking activities 940 are used to detect the smoker's progress on quitting. At least one sensor is for analyzing smoke detected for the presence of nicotine 950. If the smoker is breaking with quitting the artificial intelligence precedes with, for example, suggesting a non-nicotine non-tobacco plant-based smoking product 960 of one embodiment.

### A Cessation Digital Wearable:

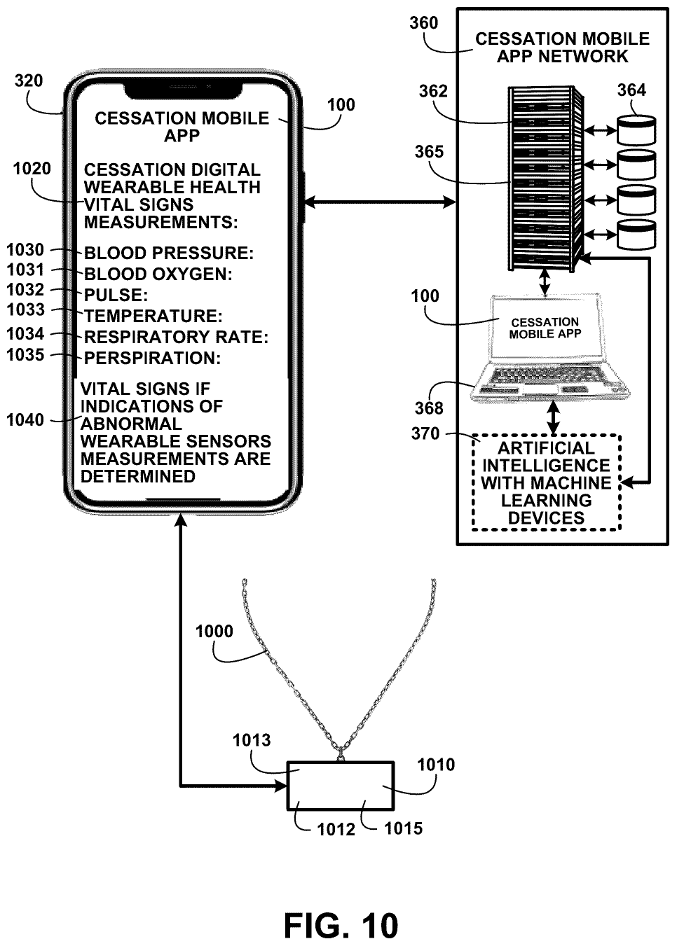

FIG. 10 shows for illustrative purposes only an example of a cessation digital wearable of one embodiment. FIG. 10 shows a user-worn pendant cessation digital wearable 1000. In one embodiment, for example the cessation digital wearable 1010 includes sensors 1015 for measuring the smoker's vital signs. The cessation digital wearable 1010 includes a wireless rechargeable battery 1012 and a wireless communication device 1013. The cessation digital wearable 1010 wireless rechargeable battery 1012 can be recharged on a wireless recharging pad when not being worn. The cessation digital wearable 1010 wireless communication device 1013 is configured for communicating to the cessation mobile app 100 installed on the smoker's cell phone over Wi-Fi, cellular, and Bluetooth.

In other embodiments, for example the cessation digital wearable 1010 with the sensors can be a badge, wrist band, an ankle bracelet, and other wearable styles in contact or close proximity to the smoker's body. In other embodiments, for example other wearable styles in contact or close proximity to the smoker's body can include headbands including sweatbands, elastic headbands, headbands worn under a hat, baseball cap, and other headgear. In yet other embodiments, for example the cessation digital wearable 1010 can be integrated into a neckerchief, bandana, scarf, necktie, leg, and cuff of a sock. In another embodiment, for example the cessation digital wearable 1010 can be an attachable device for coupling to a piece of clothing and can be worn on the inside or outside of the clothing.

The cessation digital wearable 1010 includes Wearable Health Devices (WHDs). Wearable vital signs sensing devices are miniaturizations of electronic devices used for medical detection and continuous tracking of a person's vital signs. In other embodiments, for example the cessation digital wearable 1010 is adaptable to include electrocardiogram, heart rate, blood pressure, respiration rate, blood oxygen saturation, blood glucose, skin perspiration, capnography, body temperature, motion evaluation, cardiac implantable devices, ambient parameters, and other sensors based on the smoker's particular health conditions. In one embodiment, for example a smoker with heart conditions may use a cessation digital wearable 1010 with at least an electrocardiogram sensing device with the capability of tracking the smoker's heart condition under various activities and if erratic, with the smokers pre-authorization, automatically communicate the sensor readings and smoker's identification and GPS location to the smoker's cardiologist.

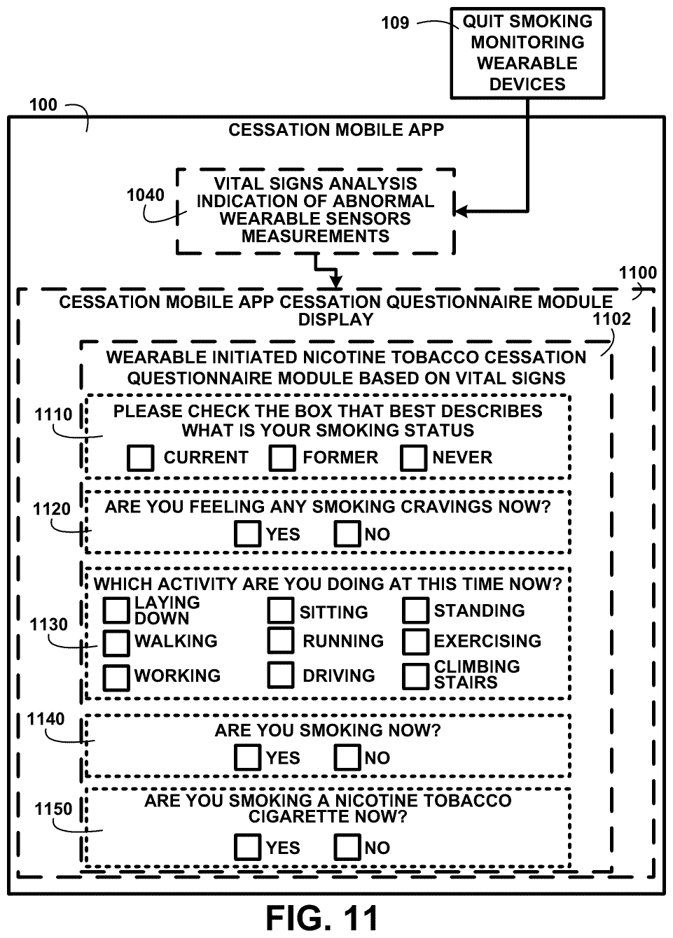

If any smoking or activity sensed that is analyzed by the cessation mobile app network 360 indicated a danger to the smoker an alert can be transmitted to the smoker's cell phone to let the smoker be aware of the danger. Vital signs indication of a detected abnormal or anomaly wearable sensors measurements 1040 will trigger an initiation of a cessation questionnaire module as described below in FIG. 11. In dire conditions, the cessation mobile app 1007 can transmit the electrocardiogram sensing device data the smoker's authorized physician and family members to alert them of the circumstance and the smoker's GPS location available from the user mobile device 320. The cessation digital wearable health vital signs measurements 1020 are displayed on the user mobile device 320 with the cessation mobile app. The cessation digital wearable health vital signs measurements: 1020 include at least blood pressure: 1030; blood oxygen: 1031; pulse: 1032; temperature: 1033; respiratory rate: 1034; and perspiration: 1035.

In one embodiment, for example the cessation digital wearable 1010 sensors 1015 include at least a humidity sensor for measuring perspiration 1035. In one embodiment, for example a cessation digital wearable 1010 blood oxygen 1031 sensor measurements can alert the smoker if low oxygen content in the blood, which can cause hypoxemia, is occurring in part due to tobacco smoke being inhaled.

The cessation digital wearable transmits electronically the sensors measured health vital signs to the user mobile device 320 with the cessation mobile app 100. The cessation mobile app 100 automatically displays the cessation digital wearable health vital signs measurements 1020. The cessation mobile app network 360 includes at least one digital server 362, plurality of databases 364, at least one digital processor 365, cessation mobile app 100 installed on a network computer 368, and artificial intelligence with machine learning devices 370. Upon receiving automatically displays the cessation digital wearable health vital signs measurements: 1020 the measurements are recorded on the plurality of databases 364.

In one embodiment, for example at least one digital processor 365 processes the recorded data to the cessation mobile app 100 installed on a network computer 368. The artificial intelligence with machine learning devices 370 analyzes the newly received data against previously recorded data for this particular smoker. In one embodiment, for example the machine learning device calculates the progress of the smoker's cessation efforts. The machine learning device also analyzes any changes in the smoker's vital signs during various activities. If the smoker has made progress in quitting smoking the positive changes in the vital signs will be recorded and displayed to the smoker. If the smoker has not fully quit smoking that analysis of the vital signs will reflect less, no improvement, and even negative results in the vital signs measurements.

The benefit to a smoker who has committed to quitting nicotine and tobacco is the supportive nature of the factual data provided with the cessation digital wearable 1010. During the various activities of the quitting committed “EX-” smoker, the display of the improvements in blood pressure: 1030, pulse: 1032, blood oxygen: 1031 levels, respiratory rate: 1034, temperature: 1033, and perspiration: 1035 start showing positive improvements daily and weekly over months and the first year. The constant improvement data supports the ex-smoker's decision to quit and reinvigorates their efforts to remain nicotine and tobacco-free of one embodiment.

### A Cessation Questionnaire Module Based on Vital Skins:

FIG. 11 shows a block diagram of an overview flow chart of a cessation questionnaire module based on vital signs of one embodiment. FIG. 11 shows the quit smoking monitoring wearable devices 109 coupled wirelessly to the cessation mobile app 100 to transmit vital signs data. The cessation mobile app 100 forwards the user's vital signs data to the cessation mobile app 100. The cessation mobile app network 360 processes the data with an analyzer. If a vital signs analysis indication of abnormal wearable sensors measurements 1040 is determined a cessation mobile app cessation questionnaire module is displayed on the user's mobile device cessation mobile app 100.

The cessation digital wearable 1010 performing the sensors 1015 of FIG. 10 vital signs measurements and transmitting the measurements to the user mobile device 320. That includes the cessation mobile app 100. The cessation mobile app 100 automatically displays vital signs measurements to the smoker. The cessation mobile app 100 automatically transmits the vital signs measurements to the cessation mobile app network 360 of FIG. 3.

The cessation mobile app network 360 of FIG. 3 records and processes the cessation digital wearable 1010 sensors 1015 of FIG. 10 vital signs measurements. The artificial intelligence with machine learning devices 370 of FIG. 3 analyzes the vital signs measurements in both normal vital signs ranges and against previously recorded vital signs measurements of the same smoker. In one embodiment, for example if vital signs indicate abnormal wearable sensors measurements 1040 the smoker is alerted. A cessation mobile app cessation questionnaire module display 1100 shows a wearable initiated nicotine tobacco cessation questionnaire module based on vital signs 1102 is displayed on the user mobile device 320 of FIG. 3. The nicotine tobacco cessation questionnaire module displays questions to the smoker for determination of the conditions that may be influencing the vital signs indication of abnormal wearable sensors measurements 1040.

In one embodiment, for example an initial question confirms the smoker's status “Please check the box that best describes what your smoking status current, former, and never” 1110. In one embodiment, for example this initial question and some other questions are in part to determine the truthfulness of the individual's answers. For example, if the initial answer is “never” but the recorded sensor data show the individual has smoked recently then the answer may be made in a defensive manner for whatever reason. The activity vital signs measurements will determine the level of truthfulness if, for example, the current pulse is equivalent to a resting pulse rate and the answer is given as “running” the individual is avoiding being truthful. At the end of the nicotine tobacco cessation questionnaire module questions, the individual will be reminded that being truthful will benefit a sincere effort to quit smoking and perhaps prevent them from developing negative health issues.

In one embodiment, for example the nicotine tobacco cessation questionnaire module questions continue with “Are you feeling any smoking cravings now?” Yes, No 1120. “Which activity are you doing at this time now?” Laying down, sitting, standing, walking, running, exercising, working, driving, and climbing stairs 1130. “Are you smoking now?” Yes, No 1140. The final question “Are you smoking a nicotine tobacco cigarette now?” Yes, No 1150. The nicotine tobacco cessation questionnaire module questions can be answered with a cell phone stylus, pencil eraser, a finger to check the box provided or verbally.

In one embodiment, for example a determination of the conditions provided in the answers that may be influencing the vital signs is made in an analysis of the recorded vital signs measurements. The determination is made with a comparison of previously recorded measurements for the same smoker during like activities. The compared measurements determine whether the current measurements are within an accepted medical range for the vital signs during like activities. Should the determination analysis show the smoker's sensed vital signs are outside the accepted medical range and the previously recorded measurements the smoker will receive both a visual and audio alert on the user mobile device 320 of the determination results. Should the determination results indicate a potentially dangerous condition to the smoker, for example, an extremely high pulse rate or low blood oxygen level the visual and audio alert will advise the smoker to stop the activity for a time and to rest until the conditions return to within the accepted medical range. In one embodiment, for example if the determination results show the smoker for instance has a pulse rate below the previously recorded measurements during the same activity and within the accepted medical range, then the visual and audio alert will convey a positive message that quitting smoking has already improved the smoker's cardio response to the activity.

The cessation digital wearable provides a supportive role in assisting a smoker to quit smoking nicotine and tobacco. The smoker wanting to quit is encouraged and supported by family and friends. The cessation digital wearable provides another level of support with factual data to demonstrate the benefits to the smoker's health. Further displaying the results of their efforts to quit on a continuous basis daily adds positive reinforcement to the resolve and emotional perspective of quitting. Many smokers develop an emotional addiction to smoking and that can be just as difficult initially to break free of as the chemical addiction to nicotine and the other harmful chemicals in tobacco. In one embodiment, for example providing cessation intervention non-nicotine non-tobacco plant-based smoking products that in appearance, taste, and smokability replace the smoker's habitual addictive nicotine tobacco product can assist in breaking the chemical addition. It makes quitting much easier by filling the emotional dependence void that frequently is the cause of a desire to relapse or fail. The additional encouragement and support provided with the cessation digital wearable system in some cases maybe the only encouragement and support for smokers with limited personal associations. Quitting smoking is difficult at best. In one embodiment, for example the combination of the non-nicotine non-tobacco plant-based smoking products and the cessation digital wearable system will help those smokers even marginally wanting to quit to overcome the difficulties and succeed in quitting nicotine and tobacco first and makes it much easier to stop the smoking habits next of one embodiment.

### Smoking Health Risks:

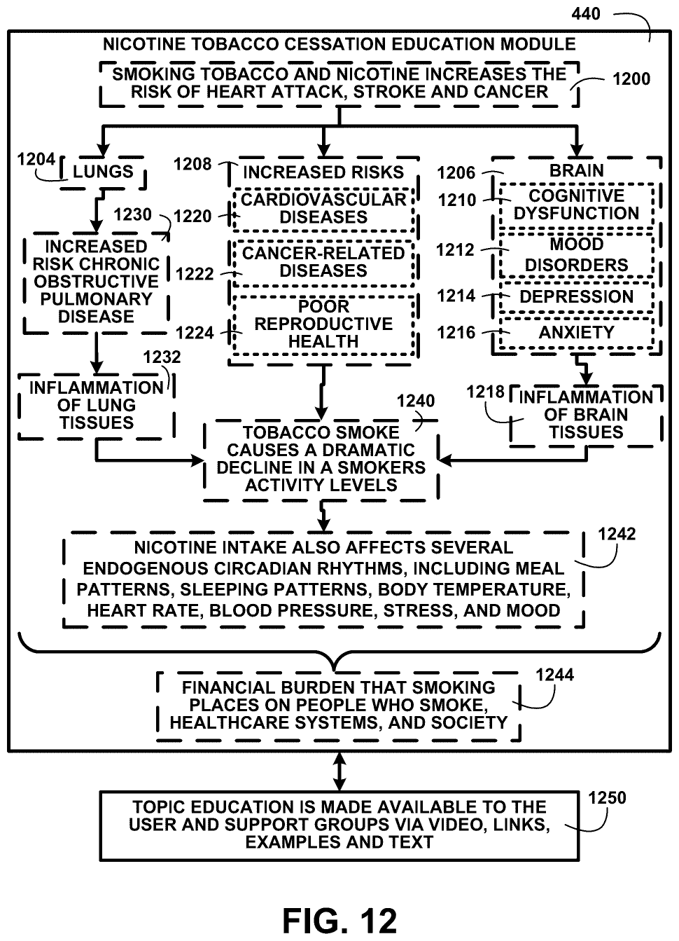

FIG. 12 shows a block diagram of an overview of smoking health risks of one embodiment. FIG. 12 shows the nicotine tobacco cessation education module 440 displaying information regarding smoking health risks. Smoking tobacco and nicotine increase the risk of heart attack, stroke, and cancer 1200. Smoking tobacco and nicotine increases damage to the brain 1206 which can include causing cognitive dysfunction 1210, mood disorders 1212, depression 1214, anxiety 1216, and inflammation of brain tissues 1218. Smoking tobacco and nicotine causes increased risks 1208 of cardiovascular diseases 1220, cancer-related diseases 1222, and poor reproductive health 1224. Smoking tobacco and nicotine increases damage to the lungs 1204 including increased risk chronic obstructive pulmonary disease 1230 and inflammation of lung tissues 1232. Tobacco smoke causes a dramatic decline in a smoker's activity levels 1240.

Nicotine intake also affects several endogenous circadian rhythms, including meal patterns, sleeping patterns, body temperature, heart rate, blood pressure, stress, and mood 1242. Also smoking tobacco and nicotine increases the financial burden that smoking places on people who smoke, healthcare systems, and society 1244 of one embodiment. All smokers need to be educated on the real health risks of their smoking habit. In one embodiment, for example the nicotine tobacco cessation education module 440 provides access to the smoker of factual data regarding the health risks in place of scare tactics some people put forth.

In some instances, the scare tactics can set-off the smoker and reinforce the attitude that someone cannot tell them they cannot do something. The factual medical and scientific data present allow the smoker to reach their own determination about quitting. The smoker can draw their own conclusions and should they elect to quit, feel at ease that they themselves reached that decision. Starting off with a self determination to quit is favorable for the smoker that they are sincere and willing to make a real effort based on real facts and a true realization of the risks and benefits to be gained. Topic education is made available to the user and support groups via video, links, examples, and text 1250.

### Smoking Disrupts Circadian Rhythms:

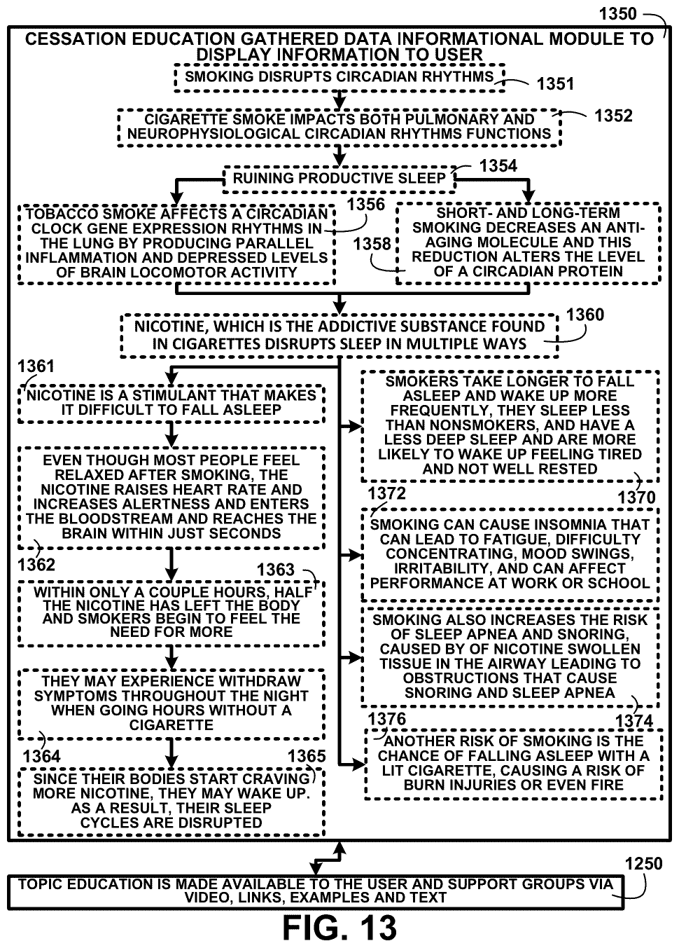

FIG. 13 shows a block diagram of an overview of smoking disrupts circadian rhythms of one embodiment. FIG. 13 shows a cessation education gathered data informational module to display information to user 1350. The information displayed shows smoking disrupts circadian rhythms 1351. Cigarette smoke impacts both pulmonary and neurophysiological circadian rhythms functions 1352. Ruining productive sleep 1354. Tobacco smoke affects a circadian clock gene expression rhythm in the lung by producing parallel inflammation and depressed levels of brain locomotor activity 1356. Short- and long-term smoking decreases an anti-aging molecule, and this reduction alters the level of a circadian protein 1358. Nicotine, which is the addictive substance found in cigarettes disrupts sleep in multiple ways 1360. Nicotine is a stimulant that makes it difficult to fall asleep 1361. Even though most people feel relaxed after smoking, the nicotine raises heart rate and increases alertness and enters the bloodstream, and reaches the brain within just seconds 1362. Within only a couple of hours, half the nicotine has left the body and smokers begin to feel the need for more 1363. They may experience withdrawal symptoms throughout the night when going hours without a cigarette 1364. Since their bodies start craving more nicotine, they may wake up, as a result, their sleep cycles are disrupted 1365. Smokers take longer to fall asleep and wake up more frequently; they sleep less than nonsmokers, have a less deep sleep, and are more likely to wake up feeling tired and not well-rested 1370. Smoking can cause insomnia that can lead to fatigue, difficulty concentrating, mood swings, irritability, and can affect performance at work or school 1372. Smoking also increases the risk of sleep apnea and snoring, caused by nicotine swollen tissue in the airway leading to obstructions that cause snoring and sleep apnea 1374. Another risk of smoking is the chance of falling asleep with a lit cigarette, causing a risk of burn injuries or even fire 1376 of one embodiment. In one embodiment, for example topic education is made available to the user and support groups via video, links, examples, and text 1250.

### Smoking Cessation Circadian Rhythms Temporary Conditions and Health Benefits:

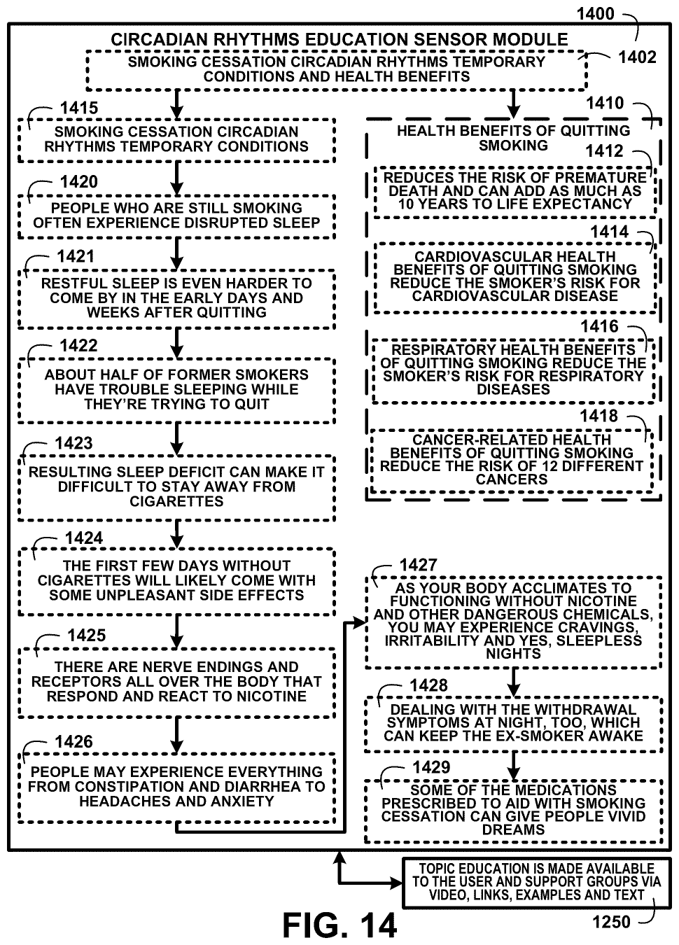

FIG. 14 shows a block diagram of an overview of smoking cessation circadian rhythms temporary conditions and health benefits of one embodiment. In one embodiment, for example FIG. 14 shows a circadian rhythms education sensor module 1400 to provide information to the user related to smoking cessation circadian rhythms temporary conditions and health benefits 1400. FIG. 14 shows the health benefits of quitting smoking 1410. Quitting smoking reduces the risk of premature death and can add as much as 10 years to life expectancy 1412. Cardiovascular health benefits of quitting smoking reduce the smoker's risk for cardiovascular disease 1414. Respiratory health benefits of quitting smoking reduce the smoker's risk for respiratory diseases 1416. Cancer-related health benefits of quitting smoking reduce the risk of 12 different cancers 1418.

Also shown are smoking cessation circadian rhythms temporary conditions 1415. Smoking disrupts circadian rhythms 1351 of FIG. 13. People who are still smoking often experience disrupted sleep 1420. Restful sleep is even harder to come by in the early days and weeks after quitting 1421. About half of former smokers have trouble sleeping while they're trying to quit 1422. The resulting sleep deficit can make it difficult to stay away from cigarettes 1423. The first few days without cigarettes will likely come with some unpleasant side effects 1424. There are nerve endings and receptors all over the body that respond and react to nicotine 1425. People may experience everything from constipation and diarrhea to headaches and anxiety 1426. As your body acclimates to functioning without nicotine and other dangerous chemicals, you may experience cravings, irritability, and yes, sleepless nights 1427. Dealing with the withdrawal symptoms at night, too, can keep the ex-smoker awake 1428. Some of the medications prescribed to aid with smoking cessation can give people vivid dreams 1429. In one embodiment, for example topic education is made available to the user and support groups via video, links, examples, and text 1250.

### Smoking Cessation Circadian Rhythms Benefits:

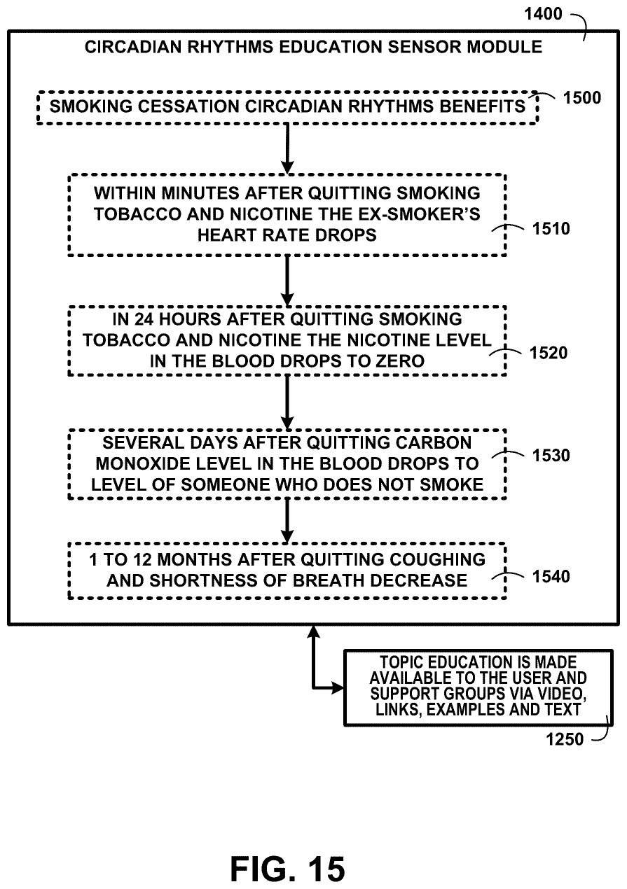

FIG. 15 shows a block diagram of an overview of smoking cessation circadian rhythms benefits of one embodiment. In one embodiment, for example FIG. 15 shows the circadian rhythms education sensor module 1400 to provide information to the user related to smoking cessation circadian rhythms benefits 1500. Within minutes after quitting smoking tobacco and nicotine the ex-smoker's heart rate drops 1510. In 24 hours after quitting smoking tobacco and nicotine, the nicotine level in the blood drops to zero 1520. Several days after quitting carbon monoxide levels in the blood drop to the level of someone who does not smoke 1530. In 1 to 12 months after quitting coughing and shortness of breath decrease 1540.

In one embodiment, for example sleep disruption caused by smoking tobacco and nicotine that may continue for a short period after quitting, but will pass and can be improved with sensor monitoring and circadian rhythms retraining of one embodiment. Providing this data of expected timelines demonstrates to the smoker that they will begin reaping benefits in much less time than they have been smoking. The initial short-term gains will encourage the smoker to stay firm in the cessation efforts. In one embodiment, for example the cessation mobile app 100 of FIG. 1 provides education on cessation medications and tools which if needed are readily available.

In one embodiment, for example the smoker will receive through the cessation mobile app recommendations and suggestions of cessation intervention products that suit their demographic and will suggest they consult with their physician especially if they have any preexisting conditions. Most smokers due to very short-term attempts to quit feel they cannot, or it will be physically and emotionally difficult. What the education provides will demonstrate the difficult episodes are very short term and there are changes in their activities that will lessen the negative effects much sooner and to a greater extent of one embodiment. Topic education is made available to the user and support groups via video, links, examples, and text 1250.

### Smoking Cessation Circadian Rhythms Monitoring:

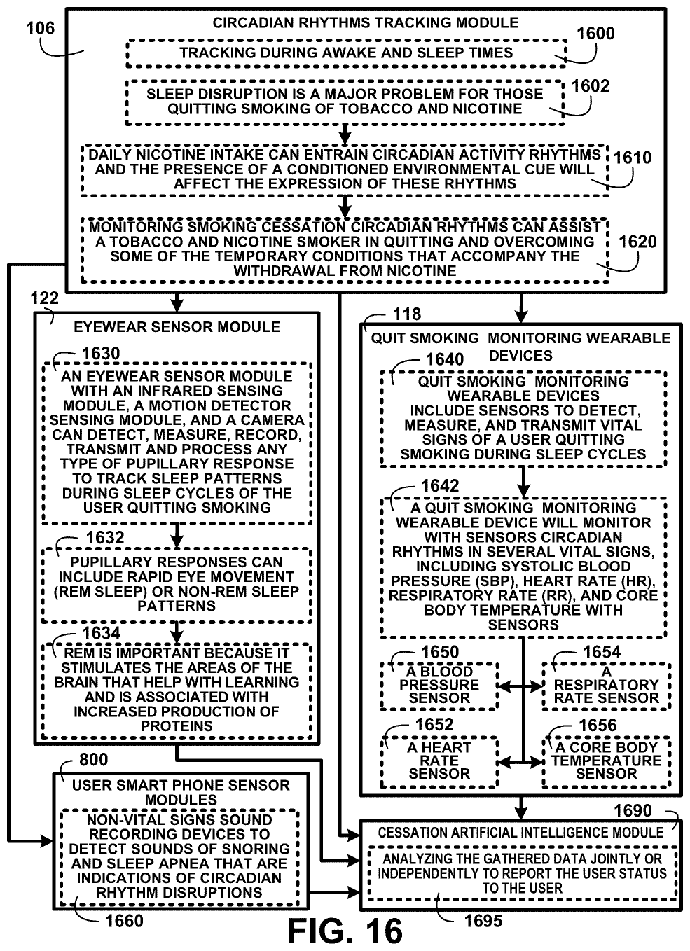

FIG. 16 shows a block diagram of an overview of smoking cessation circadian rhythms monitoring of one embodiment. In one embodiment, for example FIG. 16 shows the circadian rhythms tracking module 106 tracking a user during a sleep cycle. Sleep disruption is a major problem for those quitting smoking tobacco and nicotine 1600. Daily nicotine intake can entrain circadian activity rhythms and the presence of a conditioned environmental cue will affect the expression of these rhythms 1610. Monitoring smoking cessation circadian rhythms can assist a tobacco and nicotine smoker in quitting and overcoming some of the temporary conditions that accompany the withdrawal from nicotine 1620.

In one embodiment, for example the eyewear sensor module 122 provides visual data on the user's sleep cycle activities. An eyewear module with an infrared sensing module, a motion detector sensing module, and a camera can detect, measure, record, transmit and process any type of pupillary response to track sleep patterns during sleep cycles of the user quitting smoking 1630. Pupillary responses can include rapid eye movement (REM sleep) or non-REM sleep patterns 1632. Rem is important because it stimulates the areas of the brain that help with learning and is associated with increased production of proteins 1634.

In one embodiment, for example the quit smoking monitoring wearable devices 118 provide physiological data regarding the user during the sleep cycle. A quitting smoking wearable module includes sensors to detect, measure, and transmit vital signs of a user quitting smoking during sleep cycles 1640. A quitting smoking wearable module will monitor with sensors circadian rhythms in several vital signs, including systolic blood pressure (SBP), heart rate (HR), respiratory rate (RR), and core body temperature (T) with sensors 1642. The quitting smoking wearable module vital signs sensors include at least a blood pressure sensor 1650, a heart rate sensor 1652, a respiratory rate sensor 1654, and a core body temperature sensor 1656.

In one embodiment, for example the user smartphone sensor modules 800 provides visual data with a camera and sound data with the microphone integrated into the user smartphone. The user smartphone sensor modules 800 is also used for non-vital signs sound recording devices to detect sounds of snoring and sleep apnea that are indications of circadian rhythm disruptions 1660 of one embodiment. In one embodiment, for example the cessation artificial intelligence module 1690 is analyzing the gathered data jointly or independently to report the user status to the user 1695. This provides the smoker with frequent updates to reassure the smoker they are progressing.

### Smoking Cessation Circadian Rhythms Re-Entrainment:

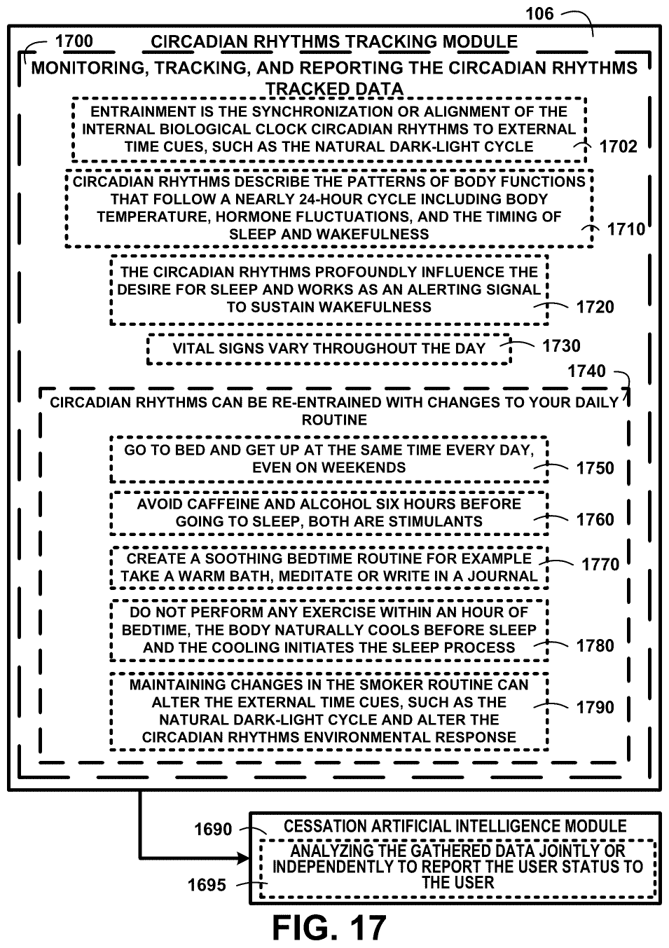

FIG. 17 shows a block diagram of an overview of smoking cessation circadian rhythms re-entrainment of one embodiment. In one embodiment, for example FIG. 17 shows the circadian rhythms tracking module 106 monitoring, tracking, and reporting the circadian rhythms tracked data 1700. Entrainment is the synchronization or alignment of the internal biological clock circadian rhythms to external time cues, such as the natural dark-light cycle 1702. Circadian rhythms describe the patterns of body functions that follow a nearly 24-hour cycle including body temperature, hormone fluctuations, and the timing of sleep and wakefulness 1710. The circadian rhythms profoundly influence the desire for sleep and works as an alerting signal to sustain wakefulness 1720. Vital signs vary throughout the day 1730.

In one embodiment, for example circadian rhythms can be re-entrained with changes to your daily routine 1740. Go to bed and get up at the same time every day, even on weekends 1750. Avoid caffeine and alcohol six hours before going to sleep, both are stimulants 1760. Create a soothing bedtime routine for example take a warm bath, meditate, or write in a journal 1770. Do not perform any exercise within an hour of bedtime, the body naturally cools before sleep and the cooling initiates the sleep process 1780. Maintaining changes in the smoker routine can alter the external time cues, such as the natural dark-light cycle, and alter the circadian rhythms environmental response 1790 of one embodiment. The cessation artificial intelligence module 1690 is analyzing the gathered data jointly or independently to report the user status to the user 1695. This provides the smoker with frequent updates to reassure the smoker they are progressing.

### Cessation of Nicotine Tobacco Smoking Habits:

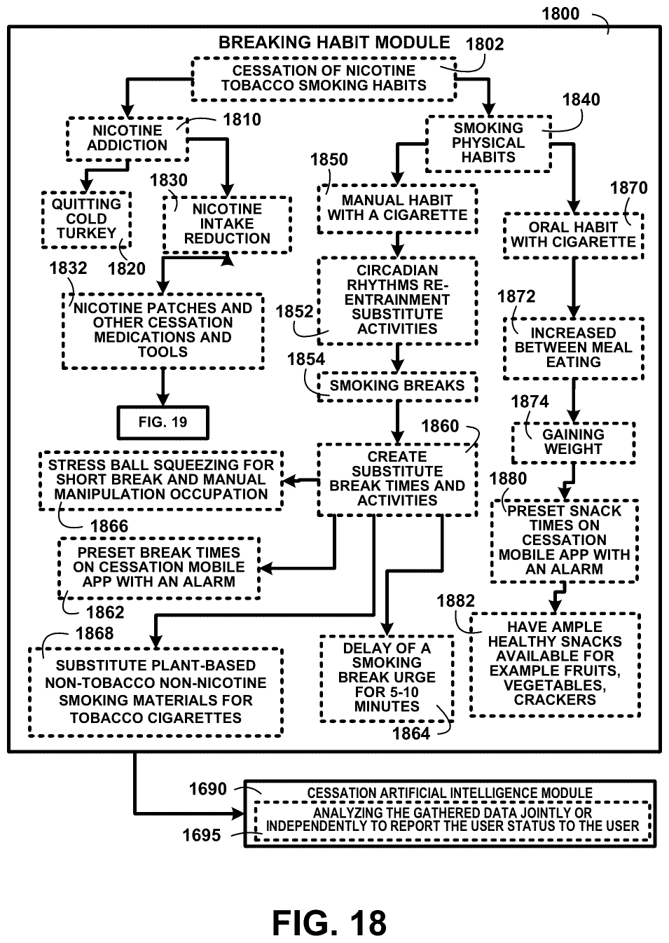

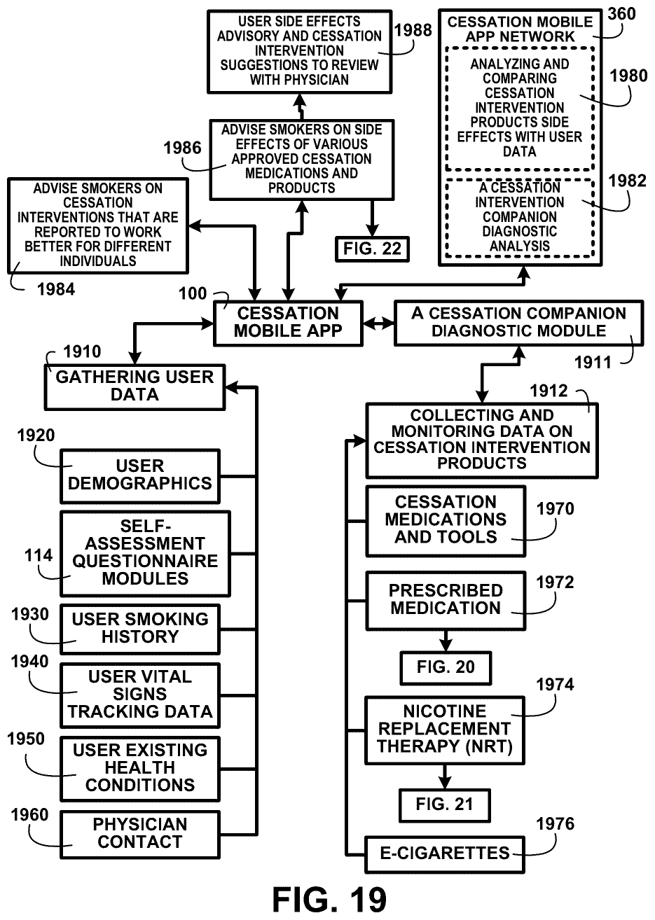

FIG. 18 shows a block diagram of an overview of cessation of nicotine tobacco smoking habits of one embodiment. In one embodiment, for example FIG. 18 shows a breaking habit module 1800 for tracking the smoker's cessation of nicotine tobacco smoking habits 1802 and making suggestions to assist the smoker. One cessation of nicotine tobacco smoking habit 1800 includes nicotine addiction 1810. Smokers may elect to try quitting cold turkey 1820 which is unaided cessation that can be supported by groups and individuals providing encouragement to the smoker through the cessation mobile app 100. In one embodiment, for example another avenue available to smokers is nicotine intake reduction 1830. This method uses, for example, nicotine patches and other cessation medications and tools 1832 that provide nicotine to the smoker in a non-smoking delivery that is gradually reduced to ween the smoker off the addictive nicotine. These medications and cessation tools are further described in FIG. 19.

In one embodiment, for example another habit to break is smoking physical habits 1840. While these habits seem less difficult than nicotine addiction, they can produce harmful outcomes that can lead the smoker to regress. One of the smoking physical habits 1840 is the manual habit with a cigarette 1850. In addition to the chemical urge for the nicotine, circadian rhythms develop for example after eating smoking that triggers the smoker to smoke after meals. The meal times trigger the manual habit to grab a cigarette and light up even though the smoker may not have the nicotine urge. In one embodiment, for example circadian rhythms re-entrainment substitute activities 1852 can assist the smoker in breaking this type of behavior of smoking breaks 1854.

Circadian rhythms re-entrainment substitute activities 1852 can be accomplished with a change in the habit to create substitute break times and activities 1860. Circadian rhythms are generally set for times of day cycles of repeating activities. In one embodiment, for example the smoker wanting to quit can preset break times on the cessation mobile app with an alarm 1862. A delay of a smoking break urge for 5-10 minutes 1864 over time will reset the circadian rhythm. In one embodiment, for example one example of a substitute activity is stress ball squeezing for a short break and manual manipulation occupation 1866.

Another of the smoking physical habits 1840 is the oral habit with cigarette 1870. An example of a smoker dealing with quitting is increased between-meal eating 1872 and the result of gaining weight 1874. In one embodiment, for example a smoker can preset snack times on the cessation mobile app with an alarm 1880 and have ample healthy snacks available for example fruits, vegetables, crackers 1882 of one embodiment. In one embodiment, for example the cessation artificial intelligence module 1690 is analyzing the gathered data jointly or independently to report the user status to the user 1695. This provides the smoker with frequent updates to reassure the smoker they are progressing.

### Cessation Intervention Companion Diagnostic Analysis:

FIG. 19 shows a block diagram of an overview of cessation intervention companion diagnostic analysis of one embodiment. In one embodiment, for example FIG. 19 shows the cessation mobile app 100 gathering user data 1910. The user data being gathered includes user demographics 1920, self-assessment questionnaire modules 114, user smoking history 1930, user vital signs tracking data 1940, user existing health conditions 1950, and physician contact 1960.

In one embodiment, for example the cessation mobile app coupled to a cessation intervention products module 1911 is using internet connections for collecting and monitoring data on cessation intervention products 1912. Cessation intervention products include cessation medications and tools 1970. These cessation medications and tools 1970 include nicotine replacement and weening delivery of reducing the amount of nicotine over a number of weeks and substitute tools to replace the tobacco nicotine cigarettes.

In one embodiment, for example a cessation companion diagnostic module coupled to the cessation mobile app continuously collects and monitors cessation intervention products data. The cessation intervention products data collected and monitored includes for example FDA Medication Guides issued with certain prescribed drugs and biological products. The information about drugs approved for human use includes at least indications and usage, dosage and administration, dosage forms and strengths, contraindications, warnings and precautions, adverse reactions, drug interactions, use in specific populations, and patient counseling information. User instructions on appropriate use of the drug, including what potential side effects may occur or follow-up that may be required as appropriate to prevent serious adverse effects.

In one embodiment, for example the use in specific populations information must include any clinically important differences in response or recommendations for use of the drug in specific populations, for example the differences between adult and pediatric responses, and a need for specific monitoring in patients with hepatic impairment. In one embodiment, for example the cessation companion diagnostic module collects and monitors information on non-prescription cessation intervention products data for example supplements, e-cigarettes and the liquids used with those tools. Instructions and directions for the use of a product are essential to its effectiveness.

In one embodiment, for example a cessation companion diagnostic module coupled to the cessation mobile app continuously collects and monitors cessation intervention products data. The cessation intervention products data collected and monitored includes for example FDA Medication Guides issued with certain prescribed drugs and biological products. The information about drugs approved for human use includes at least indications and usage, dosage and administration, dosage forms and strengths, contraindications, warnings and precautions, adverse reactions, drug interactions, use in specific populations, and patient counseling information. User instructions on appropriate use of the drug, including what potential side effects may occur or follow-up that may be required as appropriate to prevent serious adverse effects.

In one embodiment, for example the use in specific populations information must include any clinically important differences in response or recommendations for use of the drug in specific populations, for example the differences between adult and pediatric responses, and a need for specific monitoring in patients with hepatic impairment. In one embodiment, for example the cessation companion diagnostic module collects and monitors information on non-prescription cessation intervention products data for example supplements, e-cigarettes and the liquids used with those tools. Instructions and directions for the use of a product are essential to its effectiveness.

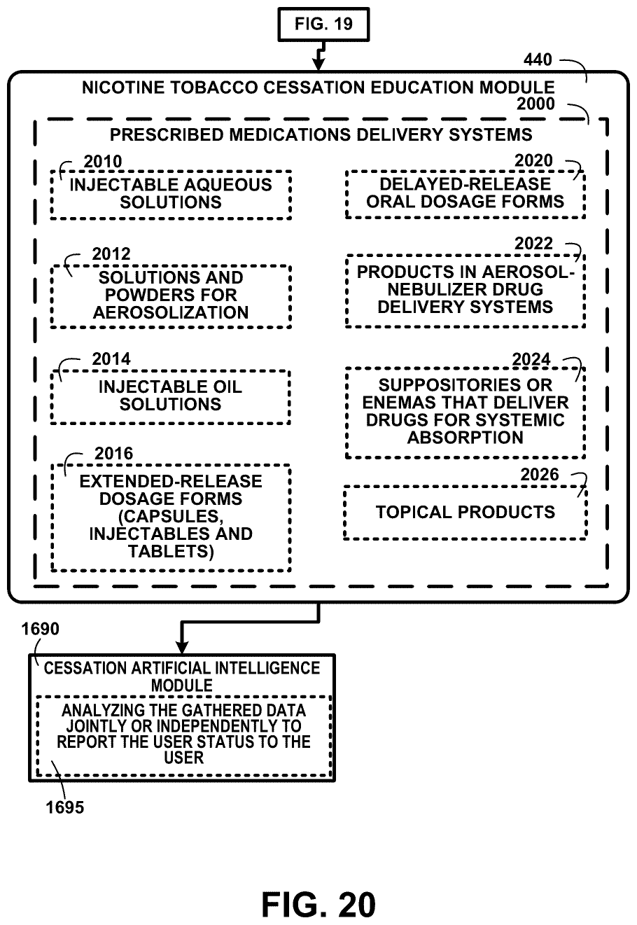

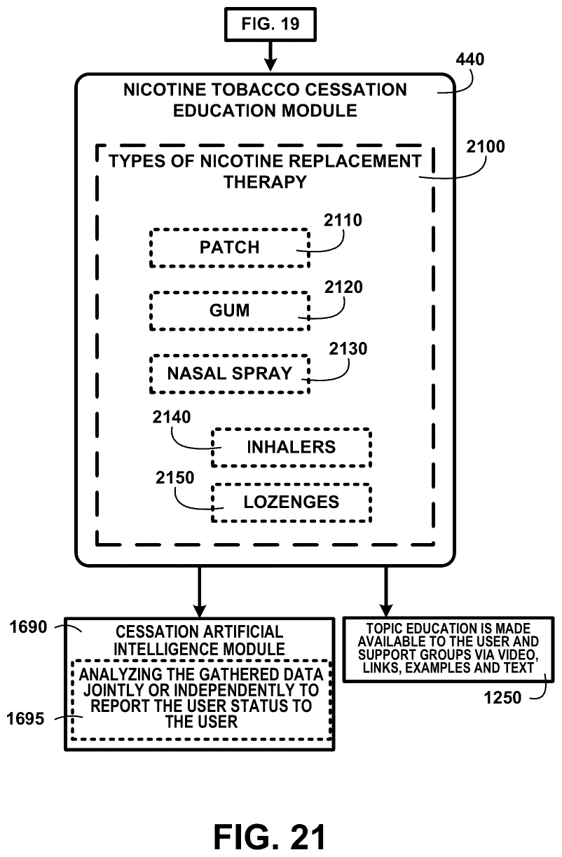

Cessation medications and tools 1970 include prescribed medication 1972 which is further described in FIG. 20. Cessation medications and tools 1970 include nicotine replacement therapy (NRT) 1974 which is further described in FIG. 21. Cessation medications and tools 1970 include e-cigarettes 1976. In one embodiment, for example the cessation mobile app network 360 uses the gathered user data and cessation medications and tools 1970 gathered data for analyzing and comparing cessation intervention products side effects with user data 1980.

In one embodiment, for example the results form a cessation intervention companion diagnostic analysis 1982. The cessation intervention companion diagnostic analysis 1982 provides suggestions to advise smokers on cessation interventions that are reported to work better for different individuals 1984. Some medications affect individuals differently than other individuals. The analysis compares the user's data to reported side effects of a person with similar traits and health conditions as the user. Any side effects that are more observed in persons sharing the user's same traits and health conditions are noted to the user.

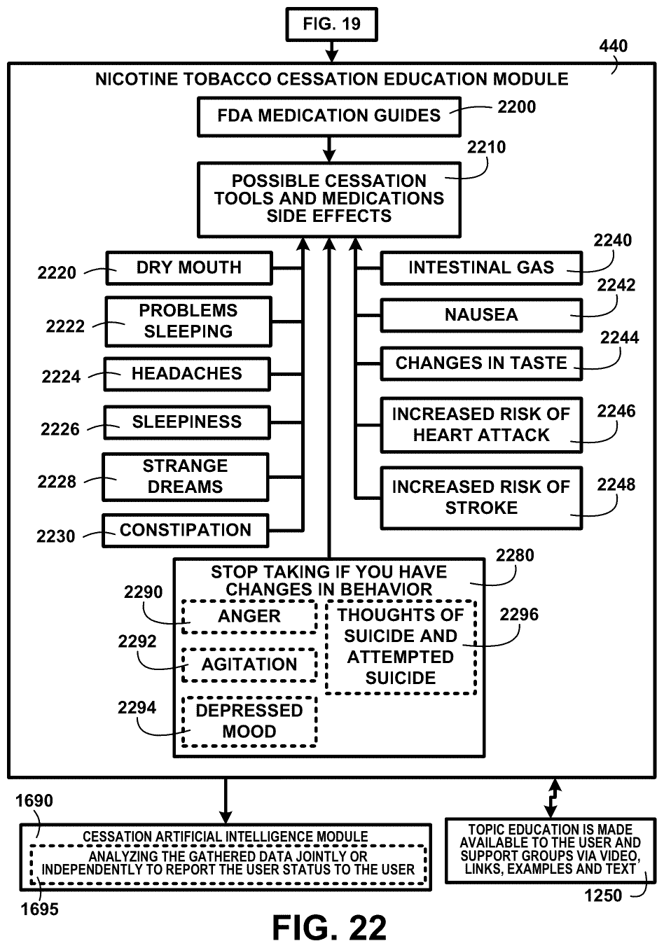

In one embodiment, for example the cessation mobile app network 360 through the cessation mobile app 100 will advise smokers on the side effects of various approved cessation medications and products 1986 as described in FIG. 22. In one embodiment, for example the user potential side effects advisory and cessation intervention suggestions to review with the user physician 1988 provide the user with appropriate warnings based on data from federal health agencies and other healthcare providers. In one embodiment, for example the suggestions may include at least one prescribed medication 1972 for which the user would have to consult with a physician to assess any side effects or dangers relating to the user's existing health conditions 1950 of one embodiment.

### Prescribed Medications Delivery Systems:

FIG. 20 shows a block diagram of an overview of prescribed medications delivery systems of one embodiment. In one embodiment, for example FIG. 20 shows a continuation from FIG. 19 with inquiries through the nicotine tobacco cessation education module 440. The nicotine tobacco cessation education module 440 in this instance is providing information on prescribed medications delivery systems 2000. The prescribed medications delivery systems 2000 information in this example include injectable aqueous solutions 2010, solutions and powders for aerosolization 2012, injectable oil solutions 2014, extended-release dosage forms (capsules, injectables and tablets) 2016, delayed-release oral dosage forms 2020, products in aerosol-nebulizer drug delivery systems 2022, suppositories or enemas that deliver drugs for systemic absorption 2024, and topical products 2026 of one embodiment. In one embodiment, for example the cessation artificial intelligence module 1690 is analyzing the gathered data jointly or independently to report the user status to the user 1695. This provides the smoker with frequent updates to reassure the smoker they are progressing.

### Types of Nicotine Replacement Therapy:

FIG. 21 shows a block diagram of an overview of types of nicotine replacement therapy of one embodiment. In one embodiment, for example FIG. 21 shows a continuation from FIG. 19 with inquiries through the nicotine tobacco cessation education module 440 for information on types of nicotine replacement therapy 2100. The types of nicotine replacement therapy 2100 in this example include patch 2110, gum 2120, nasal spray 2130, inhalers 2140, and lozenges 2150 of one embodiment. In one embodiment, for example topic education is made available to the user and support groups via video, links, examples, and text 1250. The cessation artificial intelligence module 1690 is analyzing the gathered data jointly or independently to report the user status to the user 1695. This provides the smoker with frequent updates to reassure the smoker they are progressing.

### Possible Cessation Tools and Medications Side Effects:

FIG. 22 shows a block diagram of an overview of possible cessation tools and medications side effects of one embodiment. In one embodiment, for example FIG. 22 shows a continuation from FIG. 19 with inquiries through the nicotine tobacco cessation education module 440 for information on side effects listed in FDA medication guides 2200. In one embodiment, for example the side effects information shows possible cessation tools and medications side effects 2210. The possible cessation tools and medications side effects 2210 in this example include dry mouth 2220, problems sleeping 2222, headaches 2224, sleepiness 2226, strange dreams 2228, constipation 2230, intestinal gas 2240, nausea 2242, changes in taste 2244, increased risk of heart attack 2246, and increased risk of stroke 2248. In some instances, for medications, the side effects include the advisory to stop taking if you have changes in behavior 2280 that include anger 2290, agitation 2292, depressed mood 2294, and thoughts of suicide and attempted suicide 2296 of one embodiment. In one embodiment, for example topic education is made available to the user and support groups via video, links, examples, and text 1250. The cessation artificial intelligence module 1690 is analyzing the gathered data jointly or independently to report the user status to the user 1695. This provides the smoker with frequent updates to reassure the smoker they are progressing.

### Gathering User Data:

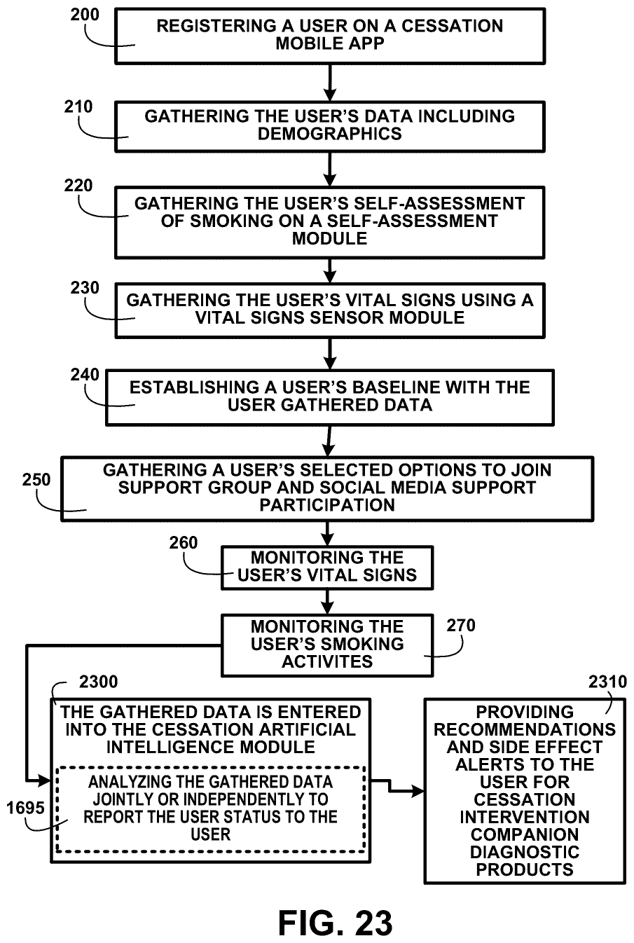

FIG. 23 shows a block diagram of an overview of gathering user data of one embodiment. In one embodiment, for example FIG. 23 shows registering a user on a cessation mobile app 200. Gathering the user data including demographics 210. Gathering the user's self-assessment of smoking on a self-assessment module 220. Gathering the user's vital signs using a vital signs sensor module 230. Establishing a user baseline with the user gathered data 240. Gathering user-selected options to join a support group and social media support participation 250. Monitoring the user's vital signs 260. Monitoring the user's smoking activities 270.

In one embodiment, for example the gathered data is entered into the cessation artificial intelligence module 2300. Analyzing the gathered data jointly or independently to report the user status to the user 1695. The cessation artificial intelligence module 2300 analysis provides data applicable to determining the smoker's progress, identifying smoking triggers, cessation impact on health conditions, and facts on any improvements in vital signs and other user activity levels. The cessation wearables are collecting data continuously and the continuing analysis make use of the data.

In one embodiment, for example the analysis is used also for making suggestions and providing education that can assist in better understanding for example nicotine withdrawal symptoms and physical habits changes to allow them to anticipate any difficult transitions and negative effects that are generally transitory. In one embodiment, for example the analysis is used also for providing recommendations and side effect alerts to the user for cessation intervention companion diagnostic products 2310 of one embodiment.

### Virtual Reality Games to Quit Smoking:

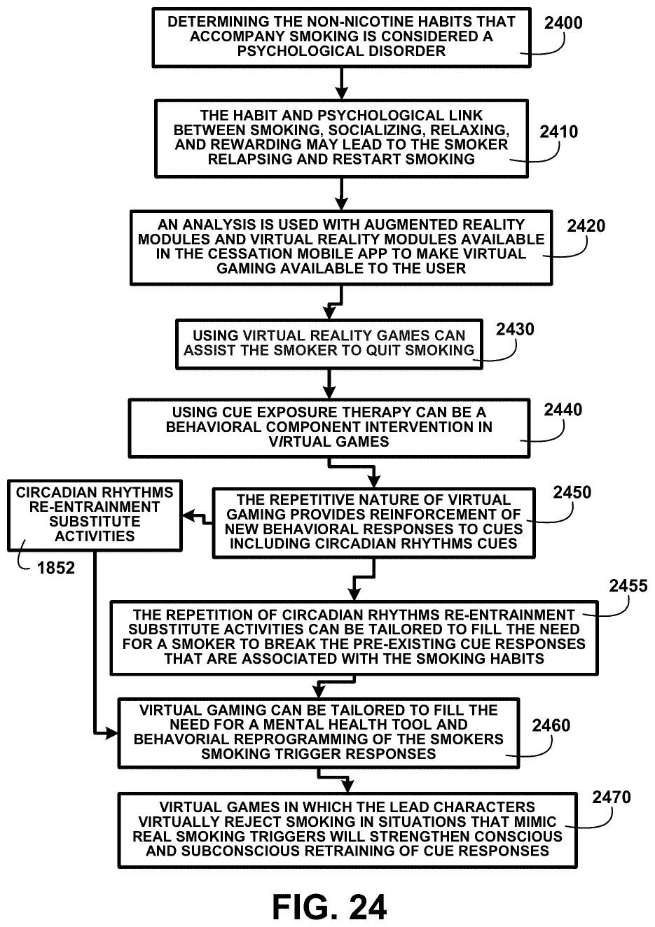

FIG. 24 shows a block diagram of an overview of virtual reality games to quit smoking of one embodiment. FIG. 24 shows the non-nicotine habits that accompany smoking is considered a psychological disorder 2400. The habit and psychological link between smoking, socializing, relaxing, and rewarding may lead to the smoker relapsing and restart smoking 2410. In one embodiment, for example the analysis is used with augmented reality modules and virtual reality modules available in the cessation mobile app to make virtual gaming available to the user 2420. Virtual reality games can assist the smoker to quit smoking 2430.

In one embodiment, for example cue exposure therapy can be a behavioral component intervention in virtual games 2440. User responses when smoking cause variations in the user's vital signs and emotional state. In one example the virtual game playing scenario is scripted to arouse anxiety in the user virtually. Anxiety in many cases is the trigger to the user smoking. The user being immersed in the virtual game environment learns how to deal with the anxiety without smoking. In time this behavioral reconditioning will be reflected in the user's vital signs data biofeedback, for example heart rate. The repetitive nature of virtual gaming provides reinforcement of new behavioral responses to cues including circadian rhythms cues 2450. The virtual gaming includes circadian rhythms re-entrainment substitute activities 1852 to replace smoking. In one embodiment, for example the repetition of circadian rhythms re-entrainment substitute activities can be tailored in the virtual game script to fill the need for a smoker to break the pre-existing cue responses that are associated with the smoking habits 2455. The virtual games features include scoring, rewards, challenges, play improvement suggestions, recognition, skill levels, leaderboards to help players improve their play and provide user motivation to continue playing the games.

In one embodiment, for example the virtual gaming includes multi-player games wherein the user, support group members and others can be playing the same game at the same time virtually. The interaction provides the user with motivation to continue with the cessation goals and achieve the non-smoking results.

In one embodiment, for example the virtual gaming can be tailored to fill the need for a mental health tool and behavioral reprogramming of the smoker's smoking trigger responses 2460. Virtual games in which the lead characters virtually reject smoking in situations that mimic real smoking triggers will strengthen conscious and subconscious retraining of cue responses 2470 of one embodiment. In one embodiment, for example virtual game development can be structured to mirror conditions confronting all smokers and can be customized for smokers of all demographics, any special health conditions, or inability due to health concerns not allowing the smoker to use a specific cessation intervention medication and tool. Long-term and heavy smokers generally have a more difficult transition away from tobacco and nicotine. A virtual game could create scenarios that fit the various circumstances. The federal health agencies indicate that 55% of smokers every year want to quit but relapse within the first year. Even though the nicotine in their systems has dissipated the smokers relapse rate indicates this is attributable to the behavioral component in quitting smoking of one embodiment.

The foregoing has described the principles, embodiments, and modes of operation of the present invention. However, the invention should not be construed as being limited to the particular embodiments discussed. The above-described embodiments should be regarded as illustrative rather than restrictive, and it should be appreciated that variations may be made in those embodiments by workers skilled in the art without departing from the scope of the present invention as defined by the following claims.

1. A cessation mobile app, comprising:
a user self-assessment processor coupled to the cessation mobile app configured to collect and record user historical and self-assessment data from user responses to questions displayed on a mobile device operating the cessation mobile application;
a vital signs processor coupled to the cessation mobile app and wirelessly coupled to app at least one user wearable health sensor configured to monitor and collect vital signs data from a user;
a cessation companion diagnostic processor coupled to the cessation mobile app configured to collect and monitor government mandated safety, efficacy and use data associated with predetermined cessation intervention products; and
an analyzer processor coupled to the cessation companion diagnostic processor configured to analyze the user historical and self-assessment data, vital signs data, and government mandated safety, efficacy and use data to rank cessation intervention products to provide customized suggestions to the user on the cessation intervention products having predetermined effectiveness for the user.

2. The cessation mobile app of claim 1, wherein at least one of the cessation intervention products includes a plant-based non-tobacco non-nicotine smoking cigarette that resembles the taste and smell of a nicotine and tobacco cigarette.

3. The cessation mobile app of claim 1, wherein the user self-assessment processor collects user data including user smoking history, the user's current smoking habit conditions and, wherein the user's self-assessment answers are configured to be entered in text and verbally, and the user's self-assessment answers are analyzed for truthfulness.

4. The cessation mobile app of claim 1, further providing at least one cessation education processor coupled to the cessation mobile app configured to provide cessation intervention products information to the user.

5. The cessation mobile app of claim 1, further comprising at least one eyewear sensor processor wirelessly coupled to the cessation mobile app configured to gather user sleep activities and transmit the gathered data to the cessation mobile app.

6. The cessation mobile app of claim 1, further comprising at least one wearable device wirelessly coupled to the cessation mobile app configured to monitor user vital signs while awake and asleep and transmit the monitored data to the cessation mobile app.

7. The cessation mobile app of claim 1, wherein the at least one cessation companion diagnostic analyzer coupled to the cessation mobile app configured to analyze cessation intervention products to advise users on cessation interventions that are reported to have predetermined effectiveness for individuals fitting the user profile.

8. A cessation mobile app comprising:
at least one self-assessment processor coupled to a cessation mobile app configured to collect and record user data as historical and self-assessment personal data and user smoking data from user responses to questions displayed on a mobile device operating the cessation mobile application;
at least one vital signs processor coupled to the cessation mobile app wirelessly coupled to at least one user wearable health sensor configured to monitor the user's vital signs while awake and asleep;
an analyzer processor coupled to a cessation companion diagnostic processor configured to analyze the user historical and self-assessment data, vital signs data, and government mandated safety, efficacy and use data to rank cessation intervention products to provide customized suggestions to the user on the cessation intervention products having predetermined effectiveness for the user;
at least one cessation companion diagnostic analyzer processor coupled to the cessation mobile app configured to collect and monitor government mandated safety, efficacy and use data associated with predetermined cessation intervention product side effects;
wherein the at least one cessation companion diagnostic analyzer processor compares the user data, vital signs data and government mandated safety, efficacy and use data to rank cessation intervention products to provide customized suggestions to on the cessation intervention products having predetermined effectiveness;
at least one analyzer processor coupled to the cessation mobile app configured to analyze collected user data;
at least one support group processor coupled to the cessation mobile app configured to create a user support group and a user social media support group;
at least one monitor processor coupled to the cessation mobile app configured to monitor collected user data related to the user's smoking activities; and
at least one alert processor coupled to the cessation mobile app configured to alert the user and support group members of user predetermined smoking trigger conditions determined from the analyzed data, and to transmit and receive support group help text messages via the user's mobile device.

9. The cessation mobile app of claim 8, further comprising at least one eyewear sensor processor wirelessly coupled to the cessation mobile app configured to gather user sleep activities and transmit the gathered data to the cessation mobile app.

10. The cessation mobile app of claim 8, further comprising at least one wearable device wirelessly coupled to the cessation mobile app configured to monitor user vital signs while awake and asleep and transmit the monitored data to the cessation mobile app.

11. The cessation mobile app of claim 8, further comprising at least one cessation education processor coupled to the cessation mobile app configured to provide cessation intervention products information to the user.

12. The cessation mobile app of claim 8, wherein cessation intervention products include cessation medications and tools including nicotine replacement medications, delivery methods of reducing amounts of nicotine and substitute tools to replace the tobacco nicotine cigarettes.

13. The cessation mobile app 8, wherein possible cessation tools and medications side effects include dry mouth, problems sleeping, headaches, sleepiness, strange dreams, constipation, intestinal gas, nausea, changes in taste, increased risk of heart attack, and increased risk of stroke.

14. The cessation mobile app of claim 8, further comprising a cessation benefits gained analysis processor coupled to the cessation mobile app configured to display beneficial user gains in health, sleep, mental health, activity levels, and attitude to encourage the user to maintain the cessation efforts.

15.-20. (canceled)

21. A cessation mobile app comprising:
a user self-assessment processor coupled to the cessation mobile app configured to collect and record user historical and self-assessment data from user responses to questions displayed on a mobile device operating the cessation mobile application;
a vital signs processor coupled to the cessation mobile app and wirelessly coupled to at least one user wearable health sensor configured to monitor and collect vital signs data from a user;
at least one support group processor coupled to the cessation mobile app configured to create a user support group and a user social media support group;
a cessation companion diagnostic processor coupled to the cessation mobile app configured to collect and monitor government mandated safety, efficacy and use data associated with predetermined cessation intervention products; and
an analyzer processor coupled to the cessation companion diagnostic processor configured to analyze the user historical and self-assessment data, vital signs data, and government mandated safety, efficacy and use data to rank cessation intervention products to provide customized suggestions to the user on the cessation intervention products having predetermined effectiveness for the user.

22. The cessation mobile app of claim 21, wherein the government mandated safety data includes side effects including dry mouth, problems sleeping, headaches, sleepiness, strange dreams, constipation, intestinal gas, nausea, changes in taste, increased risk of heart attack, and increased risk of stroke.

23. The cessation mobile app of claim 21, further comprising at least one nicotine tobacco cessation education processor configured to display on the user's mobile device and to support groups via video, links, examples, and text of medication induced changes in behavior including anger, agitation, depressed mood, and thoughts of suicide and attempted suicide.

24. The cessation mobile app of claim 21, wherein at least one of the cessation intervention products includes a plant-based non-tobacco non-nicotine smoking cigarette that resembles the taste and smell of a nicotine and tobacco cigarette.

25. The cessation mobile app of claim 21, wherein the cessation mobile app cessation self-assessment processor is further configured to transcribe the user's verbal responses to the self-assessment questions to record on the user's cell phone and transmit the transcribed responses to a network database.

26. The cessation mobile app of claim 21, further comprising the cessation mobile app displays recommendations and suggestions of cessation intervention products based on the user demographic data and suggests the user consult with their physician especially regarding any preexisting conditions.
